(19) United States
(12) Patent Application Publication
(10) Pub. No.: US 2026/0007696 A1
(43) Pub. Date: Jan. 8, 2026

(54) ORGANELLE COMPLEXES

(71) Applicant: LUCA Science Inc., Tokyo (JP)

(72) Inventors: Takahiro Shibata, Hokkaido, Sapporo (JP); Hisashi Ohta, Ibaraki, Tsukuba (JP); Keiichi Sakakibara, Hokkaido, Sapporo (JP); Yosif El-Darawish, Hokkaido, Sapporo (JP); Chia-Jung Chang, Hokkaido, Sapporo (JP); Yuya Sato, Hokkaido, Sapporo (JP); Tomoyuki Inoue, Hokkaido, Sapporo (JP); Teiji Takigawa, Hokkaido, Sapporo (JP)

(21) Appl. No.: 18/992,153

(22) PCT Filed: Jul. 6, 2023
(86) PCT No.: PCT/US2023/027014

§ 371 Date: Jan. 7, 2025

Related U.S. Application Data
(60) Provisional application No. 63/359,110, filed on Jul. 7, 2022.

(51) Int. Cl. A61K 35/12; A61P 11/00; C12N 5/00

(52) U.S. Cl. CPC A61K 35/12; A61P 11/00; C12N 5/525

(57) ABSTRACT

Disclosed herein include organelle complexes populations. The organelle complexes can comprise mitochondria and one or more of endoplasmic reticulum, peroxisomes, lysosomes, and Golgi apparatus. In some embodiments, the organelle complexes are isolated or derived from floating cells and/or frozen cells. In some embodiments, the organelle complexes are isolated or derived from cells contacted with a surfactant at a concentration at or above the critical micellar concentration (CMC) for the surfactant. At least about 80% of the mitochondria of the organelle complexes are capable of maintaining structural integrity in an extracellular environment. Also provided herein are methods for generating first organelle complexes populations.

### RELATED APPLICATIONS

This application claims the benefit under 35 U.S.C. § 119(e) of U.S. Provisional Patent Application Ser. No. 63/359,110, filed Jul. 7, 2022, the content of this related application is incorporated herein by reference in its entirety for all purposes.

### BACKGROUND

### Field

The present disclosure relates generally to methods of obtaining organelle complexes from cells, organelle complexes obtained by such methods, and uses of organelle complexes obtained by such methods.

### Description of the Related Art

Mitochondria are intracellular organelles responsible for a number of metabolic transformations and regulatory functions. They produce most of the ATP employed by eukaryotic cells. For mitochondrial function, the folded inner membrane and the surrounding outer membrane, and the electron transport system located in the inner membrane play a crucial role. The inner membrane forms a highly folded structure called cristae, which is believed to hold the supercomplex of electron transport system in the cristae membrane and to keep the proton concentration high by trapping the pumped protons in the cristae space. The electrochemical proton gradient formed by the electron transport system enables the transport of anions as well as ATP synthesis and cation transport.

Mitochondria are also highly dynamic organelles that move throughout the cell and undergo structural transitions, changing the length, morphology, shape and size. Moreover, mitochondria are continuously eliminated and regenerated in a process known as mitochondrial biogenesis. While most mitochondrial genes have been transferred to the nuclear genome, the mitochondria genome still encodes rRNAs, tRNAs, and 13 subunits of the electron transport chain (ETC). Functional communication between the nuclear and mitochondrial genomes is therefore essential for mitochondrial biogenesis, efficient oxidative phosphorylation, and normal health. Mitochondria are also the major source of free radicals and reactive oxygen species (ROS) that cause oxidative stress. Additionally, mitochondria play key roles in intracellular signaling as well as control of cell death, including apoptosis and necrosis.

There is growing evidence that mitochondrial dysfunction is associated with a broad range of human diseases. Mitochondrial dysfunction, for example, respiratory chain complex dysfunction, is a major cause responsible for a mitochondrial disease and aging. Decreased mitochondrial function influences cells in many organs principally involved in mitochondrial diseases and age-related diseases. The introduction of exogenous mitochondria into cells of a subject in need is promising approach to treating or preventing a number of diseases and disorders. However, there is a need for scalable and high surfactant-compatible methods of isolating populations comprising mitochondria from cells in a manner that retains mitochondrial function and structural integrity.

### SUMMARY

Disclosed herein include organelle complexes populations. In some embodiments, the organelle complexes are isolated or derived from floating cells and/or frozen cells. In some embodiments, the organelle complexes comprise mitochondria and one or more of endoplasmic reticulum, peroxisomes, lysosomes, and Golgi apparatus. In some embodiments, the organelle complexes are depleted of cytosolic macromolecules. In some embodiments, at least about 80% of the mitochondria of the organelle complexes maintain structural integrity in an extracellular environment.

Disclosed herein include organelle complexes populations. In some embodiments, the organelle complexes are isolated or derived from cells contacted with a surfactant at a concentration at or above the critical micellar concentration (CMC) for the surfactant. In some embodiments, the organelle complexes comprise mitochondria and one or more of endoplasmic reticulum, peroxisomes, lysosomes, and Golgi apparatus. In some embodiments, the organelle complexes are depleted of cytosolic macromolecules. In some embodiments, at least about 80% of the mitochondria of the organelle complexes maintain structural integrity in an extracellular environment.

In some embodiments, structural integrity comprises inner membrane structural integrity and/or outer membrane structural integrity of mitochondria. In some embodiments, at least 2-fold more of the mitochondria of the organelle complexes maintain structural integrity in the extracellular environment as compared to a population of homogenized mitochondria in the extracellular environment. In some embodiments, structural integrity is measured by citrate synthase (CS) activity and/or cytochrome c oxidase (COX) activity. In some embodiments, the extracellular environment comprises a total calcium concentration of about 1 to about 20 mg/dL and/or a free/active calcium concentration of about 1 to about 6 mg/dL. In some embodiments, at least about 80% of the mitochondria of the organelle complexes maintain functional capability. In some embodiments, the mitochondria of the organelle complexes are capable of ATP production. In some embodiments, the organelle complexes population comprise an at least 2-fold to 6-fold greater mitochondrial DNA (mtDNA) copy number as compared to a population of homogenized mitochondria. In some embodiments, the cytosolic macromolecules comprise cytosolic proteins, and the abundance of one or more cytosolic proteins (e.g., p70S6K and/or glyceraldehyde 3-phosphate dehydrogenase (GAPDH)) is depleted by at least about 90% as compared to the cells from which the organelle complexes population are derived.

In some embodiments, the organelle complexes comprise one or more mitochondrial matrix proteins (e.g., mitochondrial transcription factor A (TFAM), citrate synthase (CS). In some embodiments, the organelle complexes comprise one or more outer mitochondrial membrane proteins (e.g., outer mitochondrial membrane complex subunit 20 (TOMM20)). In some embodiments, the organelle complexes comprise one or more lysosome proteins (e.g., lysosomal-associated membrane protein 2 (LAMP2), mannose-6-phosphate receptor (M6PR), lysosomal-associated membrane protein 1 (LAMP1)). In some embodiments, the organelle complexes comprise: one or more peroxisome proteins (e.g., catalase, ATP-binding cassette transporter 1, subfamily D, type 3 (ABCD3)). In some embodiments, the organelle complexes comprise one or more Golgi apparatus proteins (e.g., Golgin-97, Sintaxin-6, TGOLN2/trans-Golgi network protein 2 (TGN46), Golgi matrix protein 130 (GM130), Mannosidase Alpha Class 2A Member 1 (MAN2A1)). In some embodiments, the organelle complexes comprise one or more endoplasmic reticulum proteins (e.g., Calreticulin, Calnexin).

In some embodiments, the organelle complexes population comprises first organelle complexes, or a combination of first organelle complexes and second organelle complexes. In some embodiments, first organelle complexes are derived from (i) frozen cells; (ii) floating cells; and/or (iii) cells contacted with a surfactant at a concentration at or above the critical micellar concentration (CMC) for the surfactant. In some embodiments, second organelle complexes are derived from (i) adherent cells; and/or (ii) cells contacted with a surfactant at a concentration below the critical micellar concentration (CMC) for the surfactant. In some embodiments, first organelle complexes comprise at least about 1.1-fold more of one or more lysosome proteins (e.g., lysosomal-associated membrane protein 2 (LAMP2), mannose-6-phosphate receptor (M6PR), lysosomal-associated membrane protein 1 (LAMP1)) as compared to second organelle complexes. In some embodiments, first organelle complexes comprise at least about 1.1-fold more of one or more peroxisome proteins (e.g., catalase, ATP-binding cassette transporter 1, subfamily D, type 3 (ABCD3)) as compared to second organelle complexes. In some embodiments, first organelle complexes comprise at least about 1.1-fold more of one or more Golgi apparatus proteins (e.g., Golgin-97, Sintaxin-6, TGOLN2/trans-Golgi network protein 2 (TGN46), Golgi matrix protein 130 (GM130), Mannosidase Alpha Class 2A Member 1 (MAN2A1)) as compared to second organelle complexes. In some embodiments, first organelle complexes comprise at least about 1.1-fold more of one or more endoplasmic reticulum proteins (e.g., Calreticulin, Calnexin) as compared to second organelle complexes. In some embodiments, second organelle complexes comprise at least about 1.1-fold more of one or more cytosolic proteins (e.g., p70S6K, glyceraldehyde 3-phosphate dehydrogenase (GAPDH)) as compared to first organelle complexes.

In some embodiments, upon contact of the organelle complexes with a population of cells, the organelle complexes are capable of incorporating into the cells. In some embodiments, at least about 2-fold more organelle complexes are capable of incorporating into the cells as compared to a population of homogenized mitochondria. In some embodiments, upon contact of the organelle complexes with a population of host cells, the organelle complexes have superior incorporation capability into host cells, as compared to a population of homogenized mitochondria. In some embodiments, upon contact of first organelle complexes with a population of host cells, the first organelle complexes have superior incorporation capability into host cells, as compared to second organelle complexes. In some embodiments, upon contact of the organelle complexes with a population of host cells, the organelle complexes have superior incorporation capability into host cells. In some embodiments, at least 2-fold more organelle complexes are capable of incorporation into host cells as compared to a population of homogenized mitochondria. In some embodiments, the mitochondria of the organelle complexes are capable of incorporation into cells after the population undergoes one or more freeze-thaw cycles. In some embodiments, at least 2-fold more mitochondria of the organelle complexes are capable of incorporation into cells after the population undergoes one or more freeze-thaw cycles as compared to a population of homogenized mitochondria. In some embodiments, at least about 80% of the organelle complexes in the population are between about 500 nm and about 3500 nm in size, optionally about 200 nm to about 1000 nm in size. In some embodiments, the organelle complexes population are derived from cells treated with a mitochondria-activating agent (e.g., resveratrol).

Disclosed herein include compositions comprising an organelle complexes population provided herein. Disclosed herein include formulations comprising a composition provided herein (e.g., a composition comprising an organelle complexes population) and a pharmaceutically acceptable carrier.

Disclosed herein include methods for generating first organelle complexes populations. In some embodiments, the method comprises: incubating cells in a first solution comprising a surfactant at a first temperature; removing the surfactant to form a second solution; and recovering first organelle complexes from the second solution. In some embodiments, the first organelle complexes comprise mitochondria and one or more of endoplasmic reticulum, peroxisomes, lysosomes, and Golgi apparatus. In some embodiments, the first organelle complexes population are depleted of cytosolic macromolecules. In some embodiments, (i) cells in the first solution are incubated with the surfactant at a concentration at or above the critical micellar concentration (CMC) for the surfactant; and/or (ii) the cells comprise or are derived from floating cells or frozen cells.

In some embodiments, the method comprises incubating the second solution at a second temperature. In some embodiments, the first organelle complexes comprise mitochondria and two, three, or four of endoplasmic reticulum, peroxisomes, lysosomes, and Golgi apparatus. In some embodiments, the cells in the first solution are contacted with the surfactant at a concentration at least about 5% above the critical micellar concentration (CMC) for the surfactant. In some embodiments, the surfactant is saponin. In some embodiments, the surfactant is present at a concentration of about 50 μg/mL. In some embodiments, the surfactant is a nonionic surfactant. In some embodiments, the surfactant is selected from the group consisting of Triton-X 100, Triton-X 114, Nonidet P-40, n-Dodecyl-D-maltoside, Tween-20, Tween-80, saponin, and digitonin. In some embodiments, the first solution further comprises a buffer. In some embodiments, the buffer comprises one or more of a tonicity agent, osmotic modifier, and a chelating agent. In some embodiments, the first solution comprises a Tris buffer, sucrose, and/or a chelator.

In some embodiments, incubating cells in the first solution comprises incubating the cells in the first solution for about 1 minute to about 120 minutes, optionally for about 30 minutes. In some embodiments, the first temperature and/or the second temperature is about 0° C. to about 50° C., optionally the first temperature is 25° C. and the second temperature is about 0° C. to about 4° C. In some embodiments, removing the surfactant comprises one or more washes with a buffer, optionally a Tris buffer. In some embodiments, incubating the second solution comprises incubating the second solution for about 1 minute to about 120 minutes, optionally about 20 minutes. Recovering the first organelle complexes from the second solution can comprise tangential flow filtration (TFF). TFF can be performed: (i) with a low viscosity buffer, optionally said low viscosity buffer reduces the shear rate; (ii) at about 22° C. to about 25° C.; (iii) at a shear rate less than about 2000 sec⁻¹; (iv) below room temperature, optionally at 4° C.; and/or (v) with a buffer comprising human albumin or recombinant albumin (HA). In some embodiments, recovering the first organelle complexes from the second solution comprises TFF performed using a TFF membrane with a molecular weight cutoff of at least 100 kDa, preferably of 750 kDa. In some embodiments, recovering the first organelle complexes from the second solution comprises one or more centrifugation steps. In some embodiments, recovering the first organelle complexes from the second solution comprises: centrifuging the second solution at a first centrifugal force; collecting the supernatant; centrifuging the supernatant at a second centrifugal force; and collecting the pellet to recover the first organelle complexes. In some embodiments, the first centrifugal force and/or the second centrifugal force is about 100 g to about 5000 g, optionally the first centrifugal force is about 500 g and the second centrifugal force is about 3000 g. The centrifuging can performed for about 10 minutes to about 20 minutes. In some embodiments, centrifuging the supernatant at a second centrifugal force comprises centrifuging at 8000 g for about 20 minutes.

In some embodiments, incubating cells in the first solution and/or incubating the second solution comprises applying a physical stimulus to the first solution and/or the second solution, respectively, optionally shaking and/or stirring. In some embodiments, applying a physical stimulus to the first solution and/or the second solution comprises flowing the first solution and/or the second solution through a flow device. In some embodiments said flow device comprises a fluidic channel comprising two or more segments of varying cross-sectional diameters. In some embodiments, said cross-sectional diameters range from about 0.8 mm to about 25.4 mm. In some embodiments, said flowing through the flow device generates additional flow and/or shear. In some embodiments, the method further comprises freezing the first organelle complexes, optionally in a buffer comprising a cryoprotectant. In some embodiments, said cryoprotectant comprises human albumin (HA) and/or glycerol. In some embodiments, the method further comprises treating the cells with a mitochondria-activating agent (e.g., resveratrol) prior to the incubating step. Disclosed herein include first organelle complexes populations obtained by the methods for generating first organelle complexes populations provided herein.

Disclosed herein include methods for treating a disease or disorder. In some embodiments, the method comprises: contacting cells of a subject in need thereof with an effective amount of: (i) an organelle complexes population provided herein; (ii) a composition provided herein (e.g., a composition comprising an organelle complexes population); and/or (iii) a formulation provided herein, thereby treating the disease or disorder.

Disclosed herein include methods for treating a disease or disorder associated with mitochondrial dysfunction. In some embodiments, the method comprises: contacting cells of a subject in need thereof with an effective amount of: (i) an organelle complexes population provided herein; (ii) a composition provided herein (e.g., a composition comprising an organelle complexes population); and/or (iii) a formulation provided herein, thereby treating a disease or disorder associated with mitochondrial dysfunction.

In some embodiments, contacting cells of the subject comprises a route of administration selected from the group comprising intravenous administration, intra-arterial administration, intra-tracheal administration, subcutaneous administration, intramuscular administration, inhalation, intrapulmonary administration, and intra-ocular administration. In some embodiments, the disease or disorder is selected from the group consisting of diabetes (Type I and Type II), metabolic disease, ocular disorders associated with mitochondrial dysfunction, hearing loss, mitochondrial toxicity associated with therapeutic agents, mitochondrial dysfunction associated with Space travel, cardiotoxicity associated with chemotherapy or other therapeutic agents, a mitochondrial dysfunction disorder, and migraine. In some embodiments, the disease or disorder is selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.

In some embodiments, the disease or disorder is an ischemia-related disease or disorder, a genetic disorder, an aging disease or disorder, a neurodegenerative condition, a cardiovascular condition, a cancer, an autoimmune disease, an inflammatory disease, a fibrotic disorder, or any combination thereof. In some embodiments, the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ.

In some embodiments, the neurodegenerative condition is selected from the group consisting of dementia, Friedrich's ataxia, amyotrophic lateral sclerosis, mitochondrial encephalopathy, lactic acidosis, and stroke-like episodes (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease. In some embodiments, the cardiovascular condition is selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism. In some embodiments, the disease or disorder is acute respiratory distress syndrome (ARDS) or pre-eclampsia or intrauterine growth restriction (IUGR).

### BRIEF DESCRIPTION OF THE DRAWINGS

FIGS. 1A-1B depict non-limiting exemplary data related to characterization of the organelle complexes populations provided herein. FIG. 1A depicts intracellular structures/organelles western blot protein analysis of homogenized mitochondria (H-mito), first organelle complexes (1^{st} OC), and second organelle complexes (2^{nd} OC) prepared using the methods disclosed herein from HeLa cells. FIG. 1B depicts mitochondria markers western blot protein analysis of homogenized mitochondria (H-mito), first organelle complexes (1^{st} OC), and second organelle complexes (2^{nd} OC) prepared using the methods disclosed herein from HeLa cells.

FIGS. 2A-2B depict non-limiting exemplary data related to characterization of the organelle complexes populations provided herein. FIG. 2A depicts intracellular structures/organelles western blot protein analysis of first organelle complexes (1^{st} OC) and second organelle complexes (2^{nd} OC) prepared using the methods disclosed herein from HEK293T cells. FIG. 2B depicts mitochondria markers western blot protein analysis of first organelle complexes (1^{st} OC) and second organelle complexes (2^{nd} OC) prepared using the methods disclosed herein from HEK293T cells.

FIGS. 3A-3B depict non-limiting exemplary data related to the structural integrity of first organelle complexes (1^{st} OC) and second organelle complexes (2^{nd} OC): outer membrane integrity (FIG. 3A) and inner membrane integrity (FIG. 3B).

FIG. 4 depicts non-limiting exemplary data related to the production of ATP by a first organelle complexes population at a range of Pi concentrations.

FIGS. 5A-5B depict non-limiting exemplary data related to first organelle complexes structural integrity. Citrate synthase activity (FIG. 5A) and cytochrome c oxidase activity (FIG. 5B) of first organelle complexes were assayed at a range of Ca²⁺ concentrations.

FIGS. 6A-6D depict data related to the structural integrity of first organelle complexes (1st OC) and second organelle complexes (2^{nd} OC). FIG. 6A depicts the experimental setup and FIGS. 6B-6C depict ATP production of second organelle complexes (2^{nd} OC; FIG. 6C) and first organelle complexes (1^{st} OC; FIG. 6B) at the indicated concentrations, and FIG. 6D depicts the ATP production ratio of both organelle complexes.

FIGS. 7A-7B depict the experimental setup (FIG. 7A) and data (FIG. 7B) related to the effect of second organelle complexes (2^{nd} OC) on ATP production.

FIG. 8 depicts qPCR data related to the first organelle complexes (1st OC) and second organelle complexes (2^{nd} OC) isolated from HeLa cells. Relative ratio between 1^{st} OC and 2^{nd} OC of mitochondrial DNA (mtDNA) copy number per ug protein are depicted.

FIGS. 9A-9B depict the experimental setup (FIG. 9A) and data (FIG. 9B) related to incorporation of mtDNA in recipient C6 rho0 cells incubated with homogenized mitochondria (H-mito), first organelle complexes (1^{st} OC), and second organelle complexes (2^{nd} OC). Incorporated mtDNA (FIG. 9B) 24 hours after co-incubation with homogenized mitochondria, first organelle complexes, and second organelle complexes (relative to homogenized mitochondria).

FIGS. 10A-10G depict data related to protein concentration (FIG. 10A), total ATP production (FIG. 10B), ATP production in presence of oligomycin (FIG. 10C), inner membrane integrity (FIG. 10D), outer membrane integrity (FIG. 10E), COX activity (FIG. 10F), and citrate synthase (CS) activity (FIG. 10G) of first organelle complexes recovered using final centrifugation conditions of 3000 g (10 min) or 8000 g (20 min).

FIGS. 11A-11G depict data related to a stained SDS-PAGE gel (17 μg protein/lane) (FIG. 11A) and protein levels of LAMP2 (Lysosome; FIG. 11B), Golgin97 (Golgi; FIG. 11C), Catalase (Peroxisome; FIG. 11D), Calreticulin (ER; FIG. 11E), VDAC (Mitochondria (Outer); FIG. 11F), TOMM20 (Mitochondria (Outer); FIG. 11G), and TFAM (Mitochondria (matrix); FIG. 11H) of first organelle complexes recovered using final centrifugation conditions of 3000 g (10 min) or 8000 g (20 min).

FIG. 12 depicts data related to the ATP production of fibroblasts incubated with homogenized mitochondria (H-mito), first organelle complexes, or second organelle complexes.

FIG. 13 depicts data related to first organelle complexes obtained via a first generation method (employing pipetting as a physical stimulus) versus a modified method employing a reducer flow device as a physical stimulus.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, “isolated” shall be given its ordinary meaning and shall also refer to a substance or entity that has been (1) separated from at least some of the components with which it was associated when initially produced (whether in nature or in an experimental setting), and/or (2) produced, prepared, and/or manufactured by the hand of man. In some embodiments, an isolated mitochondrion or isolated organelle complexes population has been processed to obtain it from a cellular environment via the methods provided herein.

As used herein, the term “cell” shall be given its ordinary meaning and shall also refer to a eukaryotic cell, i.e., a cell that contains mitochondria in the cytoplasm, e.g., an animal cell, e.g., a mammalian cell, preferably a human cell. As used herein, the term “cell” is used in the meaning to include a cell present in a tissue, and a cell separated from a tissue (e.g., a single cell), and a cell that is within a population of cells (e.g., a population of cells obtained from a tissue of a subject, and/or a population of cells obtained from a cell line.

As used herein, the term “mitochondrion” shall be given its ordinary meaning and shall also refer to an organelle present in a eukaryotic cell that has double-layered lipid membranes, the inner and outer membranes, and a matrix surrounded by cristae and inner membranes. Mitochondria (more than one mitochondrion) have enzymes on their inner membrane, such as the respiratory chain complexes, which is involved in oxidative phosphorylation. The inner membrane has a membrane potential due to the internal-external proton gradients formed by the action of the respiratory chain complexes, etc. Mitochondria are thought to be unable to maintain the membrane potential when the inner membrane is disrupted.

As used herein, the term “organelle complex” shall be given its ordinary meaning and shall also refer to a complex of mitochondria and one or more of endoplasmic reticulum, peroxisomes, lysosomes, and Golgi apparatus. Organelle complexes can be depleted of cytosolic macromolecules (e.g., cytosolic proteins). In some embodiments, organelle complexes do not comprise cytosolic macromolecules. In some embodiments, an organelle complexes population comprises homogenized mitochondria. As used herein, the term “population” shall be given its ordinary meaning and shall also refer to a group of a plurality of the same or different substances. For example, an “organelle complexes population” is a group of at least a plurality of the same or different organelle complexes. The population may not be always homogenous and may have physical, chemical and/or physiological distributions. The physical distribution includes, for example, particle size and polydispersity index. The chemical distribution includes, for example, a zeta potential distribution and a lipid composition distribution. The physiological distribution includes, for example, a difference of physiological function (for example, respiratory activity). An organelle complexes population can comprise first organelle complexes, second organelle complexes, homogenized mitochondria, or any combination thereof. As used herein, the term “homogenized mitochondria” shall be given its ordinary meaning and shall also refer to mitochondria isolated via a method comprising one or more homogenization steps.

As used herein, the term “surfactant” shall be given its ordinary meaning and shall also refer to a molecule having a hydrophilic moiety and a hydrophobic moiety in one molecule. Surfactants have the role of reducing surface tension at the interface or mixing polar and non-polar substances by forming micelles. Surfactants are roughly classified into nonionic surfactants and ionic surfactants. Nonionic surfactants are those in which the hydrophilic moiety is not ionized, and ionic surfactants are those in which the hydrophilic moiety comprises either a cation or an anion or both a cation and an anion.

As used herein, the term “critical micelle concentration” (CMC) shall be given its ordinary meaning and shall also refer to the concentration at which, when the concentration is reached, the surfactant forms micelles, and the surfactant further added to the system contributes to micelle formation, in particular the concentration in bulk. At concentrations above the critical micelle concentration, the addition of surfactants to the system ideally increases the amount of micelles, especially the number of micelles.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. As used herein, the term “host cell” shall be given its ordinary meaning and shall also refer to an in vivo cell, an in vitro cell, and/or an ex vivo cell into which the incorporation of exogenous mitochondria and/or organelle complexes is intended.

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “oxidative stress” shall be given its ordinary meaning and shall also refer to an imbalance between generation of reactive oxygen species, reactive nitrogen species, and/or free radicals, and the antioxidative capacity of biological system.

As used herein, the term “effective amount” refers to an amount sufficient to effect beneficial or desirable biological and/or clinical results.

The methods, compositions, systems, and kits provided herein can, in some embodiments, be employed in concert with the methods, compositions, systems, and kits described in PCT Patent Application Publication Nos. WO2018/092839, WO2017/090763, WO2020/230601, WO2019/164003, WO2020/054824, WO2020/203961, WO2020/054829, WO2021/015298, and WO2021/132735, the contents of which are incorporated herein by reference in their entirety.

### Organelle Complexes

Disclosed herein include organelle complexes populations. The organelle complexes can be isolated or derived from floating cells and/or frozen cells. The organelle complexes can be isolated or derived from cells contacted with a surfactant at a concentration at or above the critical micellar concentration (CMC) for the surfactant. The organelle complexes can comprise mitochondria and one or more of endoplasmic reticulum, peroxisomes, lysosomes, and Golgi apparatus. Disclosed herein include compositions comprising an organelle complexes population provided herein. Also disclosed herein include formulations comprising a composition provided herein (e.g., a composition comprising an organelle complexes population) and a pharmaceutically acceptable carrier. Disclosed herein also include first organelle complexes populations obtained by the methods for generating first organelle complexes populations provided herein. These first organelle complexes can be suitable for use in treatments for various diseases and disorders including those described herein, e.g., by mitochondrial transplantation. In some embodiments, organelle complexes (e.g., first organelle complexes) are internalized into cells in which mitochondria are severely dysfunctional and/or cells in which an influx of highly functional mitochondria is a benefit, to restore and/or enhance mitochondrial function.

The organelle complexes (e.g., first organelle complexes, second organelle complexes) provided herein can comprise mitochondria and one, two, three, or four of endoplasmic reticulum, peroxisomes, lysosomes, and Golgi apparatus. The organelle complexes (e.g., first organelle complexes, second organelle complexes) can comprise: (i) mitochondria and endoplasmic reticulum; (ii) mitochondria and peroxisomes; (iii) mitochondria and lysosomes; (iv) mitochondria and Golgi apparatus; (v) mitochondria, endoplasmic reticulum, and peroxisomes; (vi) mitochondria, endoplasmic reticulum, and lysosomes; (vii) mitochondria, endoplasmic reticulum, and Golgi apparatus; (viii) mitochondria, endoplasmic reticulum, peroxisomes, and lysosomes; (ix) mitochondria, endoplasmic reticulum, peroxisomes, and Golgi apparatus; (x) mitochondria, endoplasmic reticulum, peroxisomes, lysosomes, and Golgi apparatus; (xi) mitochondria, endoplasmic reticulum, lysosomes, and Golgi apparatus; (xii) mitochondria, peroxisomes, and lysosomes; (xiii) mitochondria, peroxisomes, and Golgi apparatus; (xiv) mitochondria, peroxisomes, lysosomes, and Golgi apparatus; and/or (xv) mitochondria, lysosomes, and Golgi apparatus. The ratio of mitochondria to additional organelles (e.g., endoplasmic reticulum, peroxisomes, lysosomes, and/or Golgi apparatus) in the organelle complexes population can vary. For example, in some embodiments, the molar ratio of mitochondrial proteins to proteins associated with additional organelles (e.g., endoplasmic reticulum proteins, peroxisome proteins, lysosome proteins, and/or Golgi apparatus proteins) in the organelle complexes population can be, or be about, 1:1, 1:1.1, 1:1.2, 1:1.3, 1:1.4, 1:1.5, 1:1.6, 1:1.7, 1:1.8, 1:1.9, 1:2, 1:2.5, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20, 1:21, 1:22, 1:23, 1:24, 1:25, 1:26, 1:27, 1:28, 1:29, 1:30, 1:31, 1:32, 1:33, 1:34, 1:35, 1:36, 1:37, 1:38, 1:39, 1:40, 1:41, 1:42, 1:43, 1:44, 1:45, 1:46, 1:47, 1:48, 1:49, 1:50, 1:51, 1:52, 1:53, 1:54, 1:55, 1:56, 1:57, 1:58, 1:59, 1:60, 1:61, 1:62, 1:63, 1:64, 1:65, 1:66, 1:67, 1:68, 1:69, 1:70, 1:71, 1:72, 1:73, 1:74, 1:75, 1:76, 1:77, 1:78, 1:79, 1:80, 1:81, 1:82, 1:83, 1:84, 1:85, 1:86, 1:87, 1:88, 1:89, 1:90, 1:91, 1:92, 1:93, 1:94, 1:95, 1:96, 1:97, 1:98, 1:99, 1:100, 1:200, 1:300, 1:400, 1:500, 1:600, 1:700, 1:800, 1:900, 1:1000, 1:2000, 1:3000, 1:4000, 1:5000, 1:6000, 1:7000, 1:8000, 1:9000, 1:10000, or a number or a range between any two of the values. In some embodiments, the molar ratio of mitochondrial proteins to proteins associated with additional organelles (e.g., endoplasmic reticulum proteins, peroxisome proteins, lysosome proteins, and/or Golgi apparatus proteins) in the organelle complexes population can be, or be about, 1:1, 1.1:1, 1.2:1, 1.3:1, 1.4:1, 1.5:1, 1.6:1, 1.7:1, 1.8:1, 1.9:1, 2:1, 2.5:1, 3:1, 4:1, 5:1, 6:1, 7:1, 8:1, 9:1, 10:1, 11:1, 12:1, 13:1, 14:1, 15:1, 16:1, 17:1, 18:1, 19:1, 20:1, 21:1, 22:1, 23:1, 24:1, 25:1, 26:1, 27:1, 28:1, 29:1, 30:1, 31:1, 32:1, 33:1, 34:1, 35:1, 36:1, 37:1, 38:1, 39:1, 40:1, 41:1, 42:1, 43:1, 44:1, 45:1, 46:1, 47:1, 48:1, 49:1, 50:1, 51:1, 52:1, 53:1, 54:1, 55:1, 56:1, 57:1, 58:1, 59:1, 60:1, 61:1, 62:1, 63:1, 64:1, 65:1, 66:1, 67:1, 68:1, 69:1, 70:1, 71:1, 72:1, 73:1, 74:1, 75:1, 76:1, 77:1, 78:1, 79:1, 80:1, 81:1, 82:1, 83:1, 84:1, 85:1, 86:1, 87:1, 88:1, 89:1, 90:1, 91:1, 92:1, 93:1, 94:1, 95:1, 96:1, 97:1, 98:1, 99:1, 100:1, 200:1, 300:1, 400:1, 500:1, 600:1, 700:1, 800:1, 900:1, 1000:1, 2000:1, 3000:1, 4000:1, 5000:1, 6000:1, 7000:1, 8000:1, 9000:1, 10000:1, or a number or a range between any two of the values.

The organelle complexes can be depleted of cytosolic macromolecules. Cytosolic macromolecules can be absent from the organelle complexes populations provided herein. Organelle complexes populations provided herein can comprise a negligible and/or undetectable amount of cytosolic macromolecules. The organelle complexes population can be a substantially pure organelle complexes population. A substantially pure organelle complexes population can comprise less than about 20% (e.g., less than about 20%, 18%, 16%, 14%, 12%, 10%, 8%, 6%, 4%, 2%, 1%, 0.1%, 0.01%, 0.001%, 0%, or a number or a range between any two of the values) cytosolic macromolecules. The cytosolic macromolecules can comprise cytosolic proteins, and the abundance of one or more cytosolic proteins (e.g., p70S6K and/or glyceraldehyde 3-phosphate dehydrogenase (GAPDH)) can be depleted by at least about 90% (e.g., 90%, 91%, 92%, 93%, 94%, 95%, 96%, 97%, 98%, 99%, 100%, or a number or a range between any two of these values) as compared to the cells from which the organelle complexes population are derived. The organelle complexes can comprise one or more mitochondrial matrix proteins (e.g., mitochondrial transcription factor A (TFAM), citrate synthase (CS). The organelle complexes can comprise one or more outer mitochondrial membrane proteins (e.g., outer mitochondrial membrane complex subunit 20 (TOMM20)). The organelle complexes can comprise one or more lysosome proteins (e.g., lysosomal-associated membrane protein 2 (LAMP2), mannose-6-phosphate receptor (M6PR), lysosomal-associated membrane protein 1 (LAMP1)). The organelle complexes can comprise one or more peroxisome proteins (e.g., catalase, ATP-binding cassette transporter 1, subfamily D, type 3 (ABCD3)). The organelle complexes can comprise one or more Golgi apparatus proteins (e.g., Golgin-97, Sintaxin-6, TGOLN2/trans-Golgi network protein 2 (TGN46), Golgi matrix protein 130 (GM130), Mannosidase Alpha Class 2A Member 1 (MAN2A1)). The organelle complexes can comprise one or more endoplasmic reticulum proteins (e.g., Calreticulin, Calnexin).

At least about 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the organelle complexes in the population can be between about 500 nm and about 3500 nm in size (e.g., about 500 nm, 600 nm, 700 nm, 800 nm, 900 nm, 1000 nm, 1250 nm, 1500 nm, 1750 nm, 2000 nm, 2250 nm, 2500 nm, 2750 nm, 3000 nm, 3250 nm, 3500 nm, or a number or a range between any two of these values). Organelle complexes can exhibit an about 200 nm to 1000 nm size distribution, and in some embodiments, can exhibit two peaks. In some embodiments, a first organelle complexes population exhibits a shift in size distribution (larger) relative to a second organelle complexes population. In some embodiments, the production scalability of first organelle complexes (e.g., in terms of time, reagent costs and/or labor costs) is at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) greater as compared to second organelle complexes. The organelle complexes population can comprise an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) greater mitochondrial DNA (mtDNA) copy number as compared to a population of homogenized mitochondria. A first organelle complexes population can comprise an at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) greater mitochondrial DNA (mtDNA) copy number and/or greater capacity for mtDNA incorporation into recipient cells, as compared to a second organelle complexes population.

The organelle complexes population can comprise first organelle complexes, or a combination of first organelle complexes and second organelle complexes. First organelle complexes can be derived from (i) frozen cells; (ii) floating cells; and/or (iii) cells contacted with a surfactant at a concentration at or above the critical micellar concentration (CMC) for the surfactant. Second organelle complexes can be derived from (i) adherent cells; and/or (ii) cells contacted with a surfactant at a concentration below the critical micellar concentration (CMC) for the surfactant. The organelle complexes population can be derived from cells treated with a mitochondria-activating agent (e.g., resveratrol).

There are provided, in some embodiments, second organelle complexes. In some embodiments, the method for isolating second organelle complexes from cells comprises treating cells in a first solution with a surfactant at a concentration below the critical micelle concentration (CMC) for the surfactant, removing the surfactant to form a second solution, incubating the cells in the second solution, and recovering second organelle complexes from the second solution. First organelle complexes can comprise at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of one or more lysosome proteins (e.g., lysosomal-associated membrane protein 2 (LAMP2), mannose-6-phosphate receptor (M6PR), lysosomal-associated membrane protein 1 (LAMP1)) as compared to second organelle complexes. First organelle complexes can comprise at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of one or more peroxisome proteins (e.g., catalase, ATP-binding cassette transporter 1, subfamily D, type 3 (ABCD3)) as compared to second organelle complexes. First organelle complexes can comprise at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of one or more Golgi apparatus proteins (e.g., Golgin-97, Sintaxin-6, TGOLN2/trans-Golgi network protein 2 (TGN46), Golgi matrix protein 130 (GM130), Mannosidase Alpha Class 2A Member 1 (MAN2A1)) as compared to second organelle complexes. First organelle complexes can comprise at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of one or more endoplasmic reticulum proteins (e.g., Calreticulin, Calnexin) as compared to second organelle complexes. Second organelle complexes can comprise at least about 1.1-fold (e.g., 1.1-fold, 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of one or more cytosolic proteins (e.g., p70S6K, glyceraldehyde 3-phosphate dehydrogenase (GAPDH)) as compared to first organelle complexes.

In some embodiments, at least about 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the mitochondria of the organelle complexes maintain structural integrity in an extracellular environment. Structural integrity can comprise inner membrane structural integrity and/or outer membrane structural integrity of mitochondria. In some embodiments, at least about 60%, at least about 65%, at least about 70%, at least about 75%, at least about 80%, at least about 85%, at least about 90%, at least about 95%, or more of the mitochondria of the organelle complexes have intact inner and outer membranes. In some embodiments, at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more of the mitochondria of the organelle complexes maintain structural integrity in the extracellular environment as compared to a population of homogenized mitochondria in the extracellular environment. In some embodiments, the population of the organelle complexes provided herein have the surprising feature of maintaining structural integrity and/or functional capability even when exposed to a high calcium (Ca²⁺) environment. The extracellular environment can comprise a total calcium concentration of about 1 to about 20 mg/dL and/or a free/active calcium concentration of about 1 to about 6 mg/dL. The extracellular environment may comprise a total calcium concentration of about 4 mg/dL to about 12 mg/dL, or about 1 mmol/L (1000 μM) to about 3 mmol/L (3000 μM). For example, In some embodiments, the extracellular environment comprises a concentration of total calcium of about 8 mg/dL to about 12 mg/dL, or about 2 mmol/L (2000 μM) to about 3 mmol/L (3000 μM). In some embodiments, the extracellular environment comprises a concentration of free or active calcium of about 4 mg/dL to about 6 mg/dL, or about 1 mmol/L (1000 PM) to about 1.5 mmol/L (1500 μM). In some embodiments, mitochondria of the organelle complexes maintain functional capability in an environment having a higher calcium concentration compared to the calcium environment in a cell. Thus, in some embodiments, the mitochondria of the organelle complexes provided herein possess the remarkable characteristics of being isolated from a cellular environment (including from frozen cells or floating cells) with minimal or negligible damage, and retain capacity to function even when exposed to an extracellular environment, e.g., a calcium rich environment that would otherwise be expected to cause damage to the mitochondria and/or significantly inhibit their functional capacity.

In some embodiments, inner membrane structural integrity and/or outer membrane structural integrity (e.g., the presence of intact inner and/or outer membranes) can be determined by the functional activity of the mitochondria, for example, the membrane potential and polarization. Structural integrity can be measured by citrate synthase (CS) activity and/or cytochrome c oxidase (COX) activity. In some embodiments, at least about 80% (e.g., 80%, 81%, 82%, 83%, 84%, 85%, 86%, 87%, 88%, 89%, 90%, 91%, 92%, 93%, 94%, 95%, 96%, 97%, 98%, 99%, 100%, or a number or a range between any two of these values) of the mitochondria of the organelle complexes maintain functional capability. The mitochondria of the organelle complexes can be capable of ATP production in some embodiments provided herein. ATP production of an organelle complexes population provided herein can, in some embodiments, exceed the ATP production of homogenized mitochondria by at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values). In some embodiments, the functional capability in an extracellular environment is measured by a fluorescence indicator of membrane potential. In some embodiments, the fluorescence indicator is selected from positively charged dyes such as JC-1, TMRM, and TMRE.

Organelle complexes population may have various properties that facilitate delivery of a payload, such as, a desired transgene or exogenous agent, to a target cell. The transgene can encode a therapeutic protein. The exogenous agent can be selected from the group comprising a nucleic acid molecule, a protein or polypeptide, a small molecule, a hormone, and any combination thereof. The exogenous agent can comprise or be a viral vector, bacterial vector, plasmid vector, or any combination thereof. In some embodiments, the exogenous agent comprises a nucleic acid molecule selected from the group consisting of a ribonucleic acid, small RNA molecule, complementary RNA, a non-coding RNA molecule, siRNA, a pi-RNA molecule, a micro-RNA molecule, a sno-RNA molecule, long non-coding RNA molecule, messenger RNA molecule, ribosomal RNA molecule, an antisense nucleic acid molecule, Locked Nucleic Acid (LNA), antagomir, CRISPR/Cas gene editing RNA, trans-activating crRNA (tracrRNA), short synthetic RNA composed of a “scaffold” sequence (gRNA), Small Cajal body-specific RNAs (scaRNA), natural cis-antisense siRNAs (cis-nat-siRNAs), trans-acting siRNA (tasiRNA), repeat associated small interfering RNA (rasiRNA), 7SK, transfer-messenger RNA (tmRNA), transfer RNA (tRNA), 7SL RNA, signal recognition particle RNA (SRP), or any combination thereof.

Upon contact of the organelle complexes with a population of cells, the organelle complexes provided herein can be capable of incorporating into the cells. In some embodiments, incorporation into a cell comprises colocalization and/or fusion with endogenous mitochondria within said cell. The cells can be in vivo, in vitro, or ex vivo. In some embodiments, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more organelle complexes are capable of incorporating into the cells as compared to a population of homogenized mitochondria. Upon contact of the organelle complexes with a population of host cells, the organelle complexes can have superior incorporation capability into host cells, as compared to a population of homogenized mitochondria. Upon contact of first organelle complexes with a population of host cells, the first organelle complexes can have superior incorporation capability into host cells, as compared to second organelle complexes. In some embodiments, at least about 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more first organelle complexes are capable of incorporating into the cells as compared to second organelle complexes. Upon contact of the organelle complexes with a population of host cells, the organelle complexes can have superior incorporation capability into host cells. In some embodiments, at least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more organelle complexes are capable of incorporation into host cells as compared to a population of homogenized mitochondria. In some embodiments, and without being bound by any particular theory, the superior incorporation capacity of the organelle complexes provided herein are responsible, at least in part, for the superior clinical effect exhibited by said organelle complexes when used to treat any diseases or disorders such as those described herein.

In some embodiments, the organelle complexes population provided herein are capable of being incorporated into cells after storage of the mitochondria at any temperature provided herein (e.g., 4° C.±3° C., −20° C.±3° C., −80° C.±3° C., or in liquid nitrogen). The mitochondria of the organelle complexes can be capable of incorporation into cells after the population undergoes one or more freeze-thaw cycles. At least 2-fold (e.g., 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) more mitochondria of the organelle complexes can be capable of incorporation into cells after the population undergoes one or more freeze-thaw cycles as compared to a population of homogenized mitochondria.

### Methods of Generating First Organelle Complexes Populations

Disclosed herein include methods for generating first organelle complexes populations. In some embodiments, the method comprises: incubating cells in a first solution comprising a surfactant at a first temperature; removing the surfactant to form a second solution; and recovering first organelle complexes from the second solution. The first organelle complexes can comprise mitochondria and one or more of endoplasmic reticulum, peroxisomes, lysosomes, and Golgi apparatus. The first organelle complexes population can be depleted of cytosolic macromolecules. In some embodiments, (i) cells in the first solution are incubated with the surfactant at a concentration at or above the critical micellar concentration (CMC) for the surfactant; and/or (ii) the cells comprise or are derived from floating cells or frozen cells.

There are provided, in some embodiments, methods of generating first organelle complexes. The generation of first organelle complexes can comprise: (Step A) providing adherent, floating, and/or frozen cells, thawing, and placing in a tube. The generation of first organelle complexes can comprise: (Step A) providing adherent, floating, and/or frozen cells, centrifuging, and collecting the precipitant. The generation of first organelle complexes can comprise: (Step A) providing adherent cells, aspirating, adding a solution (e.g., PBS(−)), aspirating, adding TrypLE, incubating, adding a solution (e.g., PBS(−)), placing the cell suspension in a tube, centrifuging, and collecting the precipitant. The generation of first organelle complexes can comprise one or more of the following steps: (Step B) adding Tris Buffer, centrifuging, and collecting the precipitant; (Step C) adding Tris Buffer and vortexing; (Step D) adding a solution comprising a surfactant and incubating; (Step E) centrifuging and collecting the precipitant; (Step F) adding Tris Buffer, centrifuging and collecting the precipitant; (Step G) adding Tris Buffer and pipetting; (Step H) transferring to another tube and collecting buffer solution in the original tube and rinsing it; (Step I) centrifuging and collecting the supernatant; (Step J) centrifuging and collecting the precipitant; and (Step K) pipetting One or more of the above steps can comprise an incubation period. One or more of the above steps can comprise a centrifugation step, followed by collection of the supernatant and/or the precipitant. One or more of the above steps can be omitted and one or more additional steps can be included. The times, volumes, concentrations, and centrifugal forces can vary depending on the embodiment.

In some embodiments, the cells employed in the disclosed methods for generating first organelle complexes populations can be adherent cells, floating cells, detached cells, suspension cells, frozen cells, or any combination thereof. The cells can be in the form of cells present in a tissue, or they may be isolated from a tissue (e.g., single cells) or a population thereof. The cells isolated from the tissue may be cultured cells, or single cells or a population thereof, obtained by treatment of the tissue or cultured cells with enzymes used to make them be single cells, such as collagenase. Tissues may be chopped, if desired, prior to enzymatic treatment, such as collagenase. The method further can comprise treating the cells with a mitochondria-activating agent (e.g., resveratrol) prior to the step of incubating cells in the first solution. First organelle complexes can be derived from cells wherein the mitochondria are activated. Activation of mitochondria can be achieved by various methods, for example, by contacting the mitochondria with a mitochondria activating agent. Such an activation of mitochondria can be achieved by various methods including MITO-Porter technology. MITO-Porter technology may use a complex of mitochondria targeting carrier and a mitochondria activating agent. As used herein, the term “mitochondria activating agent” can refer to a substance capable of activating a mitochondrial respiratory chain complex (electron transport system), particularly a substance capable of bringing mitochondria into a polarized state in terms of a membrane potential, and in particular, it is preferable to use a substance capable of bringing mitochondria into a hyperpolarized state. Examples of the mitochondria activating agent may include antioxidants such as resveratrol (3,5,4′-trihydroxy-trans-stilbene), coenzyme Q10, vitamin C, vitamin E, N-acetylcysteine, 2, 2,6,6, -tetramethylpiperidine 1-oxyl (TEMPO), superoxide dismutase (SOD) and glutathione, and in particular, resveratrol is preferable (see WO2018/092839). Other examples of mitochondria activating agent include mitochondria DNA, and mitochondria RNA such as 12S rRNA and 16S rRNA (see WO2020/230601, which is incorporated herein by reference in its entirety), and any other component of mitochondria.

Step A can comprise adding about 1 mL to about 10 mL of a solution (e.g., PBS(−)). Step A can comprise adding about 1 mL to about 5 mL of TrypLE. Step A can comprise incubating at about 30° C.-40° C. for about 2 min to about 10 min. Step A can comprise thawing at a temperature of about 30° C.-42° C. for about 1 min to about 7 min. Step A can comprise employing adherent cells from a dish that is about 5 cm to about 20 cm. Step A, Step B, Step E, and/or Step I can comprise centrifuging at about 200 g to about 800 g for about 5 min to about 20 min at about 0° C.-10° C. Step A and/or Step H can comprise using a tube that is about 10 mL to about 75 mL. Step B, Step C, Step F, and/or Step G can comprise adding about 0.5 mL to about 6 mL of Tris Buffer. Step C can comprise vortexing for about 5 sec to about 20 sec. Step D can comprise adding about 0.5 mL to about 2 mL of a solution comprising the surfactant. The surfactant can be present at a concentration of about 50 μg/mL to about 200 μg/mL in the solution comprising the surfactant. The final concentration of the surfactant following addition in Step D can be about 25 μg/mL to about 75 μg/mL. Step D can comprise incubating at about 18° C.-28° C. for about 20 min to about 40 min. Step F can comprise centrifuging at about 500 g to about 1500 g for about 1 min to about 10 min at about 0° C.-10° C. Step G can comprise incubating at about 0° C.-5° C. for about 10 min to about 30 min. Step G and/or Step K can comprise pipetting about 5 times to about 30 times. Step H can comprise collecting about 0.5 mL to about 2 mL of buffer solution in the original tube and rinsing it. Step J can comprise centrifuging at about 2000 g to about 4000 g for about 5 min to about 20 min at about 0° C.-10° C. Step J can comprise centrifuging at about 6000 g to about 10000 g for about 10 min to about 30 min at about 0° C.-10° C. Collecting the precipitate can comprise removing supernatant (e.g., 0.5 mL to about 2.5 mL).

There are provided, in some embodiments, first generation methods (e.g., centrifugation-based methods) of generating first organelle complexes that can comprise some or all of the steps provided above (e.g., Step A, Step B, Step C, Step D, Step E, Step F, Step G, Step H, Step I, Step J, and/or Step K). There are provided, in some embodiments, second generation methods (e.g., a TFF method) of generating first organelle complexes that can comprise substitutions of (and/or additions to) one or more of Step A, Step B, Step C, Step D, Step E, Step F, Step G, Step H, Step I, Step J, and/or Step K. For example, in some embodiments, cell washing/preparation can comprise the use of a Counterflow Centrifugation System (e.g., Rotea). For example, saponin treatment and/or first organelle complexes extraction can comprise the use of a flow device (e.g., reducer flow device). Said flow device can comprise a fluidic channel comprising two or more segments of varying cross-sectional diameters In some embodiments, and without being bound by any particular theory, flow through a reducer flow device can cause changes in flow velocity due to changes in flow cross-section areas. Reducer flow devices provided herein can have various configurations, such as, for example, square reducers, tapered reducers, concentric reducers, and/or eccentric reducers. The flow device can comprise various types and sizes of tubes to create additional flow and shear for first organelle complexes extraction. Lysate polishing can comprise prefiltering and/or Rotea. Tangential Flow Filtration (TFF) can be employed for purification and/or buffer exchange in some embodiments of second generation methods (e.g., a TFF method) provided herein. Step B can comprise the use of Rotea. Step G can comprise the use of a flow device (e.g., reducer flow device). Step I can comprise cell lysate polishing (e.g., prefilter or Rotea or combination). Step J can comprise TFF. Step K can comprise the use of cryoprotectants comprising human albumin (HA) and/or glycerol. The use of HA and/or glycerol in preservation buffers can enhance the stability of first organelle complexes during storage at low temperatures (e.g, −80° C.) and can improve their quality following thawing.

The cells in the first solution can be contacted with the surfactant at a concentration at least about 5% (e.g., 5%, 6%, 7%, 8%, 9%, 10%, 11%, 12%, 13%, 14%, 15%, 16%, 17%, 18%, 19%, 20%, 25%, 30%, 40%, 50%, 75%, 100%, or higher and overlapping ranges therein) above the critical micellar concentration (CMC) for the surfactant. The surfactant can be saponin and/or digitonin. The surfactant can be present at a concentration of about 50 μg/mL. The surfactant can be a nonionic surfactant. The surfactant can be selected from the group consisting of Triton-X 100, Triton-X 114, Nonidet P-40, n-Dodecyl-D-maltoside, Tween-20, Tween-80, saponin, and digitonin.

In some embodiments, the surfactant used in the methods provided herein may be an ionic or a nonionic surfactant. Nonionic surfactants used in this disclosure may include, for example, ester, ether, and alkyl glycoside forms. Non-ionic surfactants include, for example, alkyl polyethylene glycols, polyoxyethylene alkylphenyl ethers, and alkyl glycosides. Nonionic surfactants may include Triton-X 100, Triton-X 114, Nonidet P-40, n-Dodecyl-D-maltoside, Tween-20, Tween-80, saponin and/or digitonin. The concentration of surfactant(s) present in the first solution can be, can be about, can be at least, or can be at most, 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800, 810, 820, 830, 840, 850, 860, 870, 880, 890, 900, 910, 920, 930, 940, 950, 960, 970, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3250, 3500, 3750, 4000, 4250, 4500, 4750, 5000, 5500, 6000, 6500, 7000, 7500, 8000, 8500, 9000, 9500, 10000, or a number or a range between any two of these values, μg/mL or μM.

In some embodiments, the first solution and/or the second solution can comprise a buffer. The buffer can comprise one or more of a tonicity agent, osmotic modifier, and a chelating agent. The first solution and/or the second solution can comprise a Tris buffer, sucrose, and/or a chelator. Exemplary buffers for use in the methods provided herein include, for example, Tris buffer, HEPES buffer, and phosphate buffer. Buffers may be, for example, pH 6.7-7.6 (e.g., pH 6.8-7.4, pH 7.0-7.4, e.g., pH 7.2-7.4, e.g., pH 7.4). In some embodiments, the buffers may include tonicity agents and osmotic modifiers. Exemplary tonicity agents and osmotic modifiers include monosaccharides (e.g., glucose, galactose, mannose, fructose, inositol, ribose, xylose, etc.), disaccharides (e.g., lactose, sucrose, cellobiose, trehalose, maltose, etc.), trisaccharides (e.g., raffinose, melesinose, etc.), polysaccharides (e.g., cyclodextrin, etc.), sugar alcohols (e.g., erythritol, xylitol, sorbitol, mannitol, maltitol, etc.), glycerin, diglycerin, polyglycerin, propyleneglycol, polypropyleneglycol, ethyleneglycol, diethyleneglycol, triethyleneglycol, polyethyleneglycol, and the like. Buffers may also contain a chelating agent, particularly a chelating agent for divalent metals, such as a chelating agent for calcium ion. Chelating agents include, for example, glycol ether diaminetetraacetic acid (EGTA) and ethylenediaminetetraacetic acid (EDTA).

The method can comprise incubating the second solution at a second temperature. The first temperature and/or the second temperature can be about 0° C. to about 50° C. The first temperature can be 25° C. and the second temperature can be about 0° C. to about 4° C. The first temperature and/or second temperature can be, can be about, can be at least, or can be at most, 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or a number or a range between any two of these values. Removing the surfactant can comprise one or more washes with a buffer (e.g., a Tris buffer). One or more steps of the methods provided herein can be carried out on ice or at room temperature.

Incubating cells in the first solution can comprise incubating the cells in the first solution for about 1 minute to about 120 minutes, optionally for about 30 minutes. Incubating the second solution can comprise incubating the second solution for about 1 minute to about 120 minutes, optionally about 20 minutes. The step of incubating cells in the first solution and/or the step of incubating the second solution can comprise a period of time of about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values, minutes. Incubating cells in the first solution and/or incubating the second solution can comprise applying a physical stimulus to the first solution and/or the second solution, respectively, such as, for example, pipetting, shaking and/or stirring. Applying a physical stimulus to the first solution and/or the second solution can comprise flowing the first solution and/or the second solution through a flow device (e.g., a reducer flow device). Said flow device can comprise a fluidic channel comprising two or more segments of varying cross-sectional diameters. Said cross-sectional diameters can be, can be about, can be at least, or can be at most, 0.1 mm, 0.2 mm, 0.3 mm, 0.4 mm, 0.5 mm, 0.6 mm, 0.7 mm, 0.8 mm, 0.9 mm, 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, or a number or a range between any two of these values. In some embodiments, said flowing through the flow device generates additional flow and/or shear.

Removing the surfactant to form a second solution can comprise decreasing the concentration of surfactant in the solution in which the first organelle complexes come into contact, including, for example, less than 10%, less than 5%, less than 4%, less than 3%, less than 2%, or less than 1% or less of the concentration of surfactant; or below the detection limit in the solution in which the first organelle complexes come into contact. To ensure removal of the surfactant from the solution, removing the surfactant to form a second solution may include washing the cells with a solution containing a lower or reduced concentration of surfactant (preferably a surfactant-free solution) (e.g., a buffer). In order to remove the surfactant from the first solution, the solution added to or exchanged with the first solution can be a buffer and, in some embodiments, is the buffer employed in the first solution (but a solution containing a lower concentration of surfactant, preferably a solution with no surfactant or undetectable levels of surfactant). The second solution can be a solution containing a lower concentration of surfactant. In some embodiments, the second solution is a surfactant-free solution or a solution with a negligible and/or undetectable amount of surfactant. The solution used in the recovering step (e.g., the second solution) may be a solution comprising, for example, a buffer, an osmotic modifier, and a divalent metal chelator, substantially free of surfactants. As used herein, “substantially free” is used in the sense of not excluding the presence of contamination with an amount of “substantially free ingredient” that cannot be removed or cannot be detected.

In some embodiments, recovering the first organelle complexes comprises application of one or more physical stimulus to the second solution. The recovering step can comprise incubating the second solution at a second temperature. Thus, the recovering step can be carried out under shaking or non-shaking conditions. The incubation of the recovering step can be carried out under stirring or non-stirring conditions. First organelle complexes can be collected as a precipitate by subjecting the second solution to one or more centrifugation steps. Recovering the first organelle complexes from the second solution can comprise one or more centrifugation steps. Recovering the first organelle complexes from the second solution can comprise: centrifuging the second solution at a first centrifugal force; collecting the supernatant; centrifuging the supernatant at a second centrifugal force; and collecting the pellet to recover the first organelle complexes. The first centrifugal force and/or the second centrifugal force can be about 100 g to about 5000 g. The first centrifugal force can be about 500 g and the second centrifugal force can be about 3000 g. The first centrifugal force and/or the second centrifugal force can be, can be about, can be at least, or can be at most, 100 g, 110 g, 120 g, 128 g, 130 g, 140 g, 150 g, 160 g, 170 g, 180 g, 190 g, 200 g, 210 g, 220 g, 230 g, 240 g, 250 g, 260 g, 270 g, 280 g, 290 g, 300 g, 310 g, 320 g, 330 g, 340 g, 350 g, 360 g, 370 g, 380 g, 390 g, 400 g, 410 g, 420 g, 430 g, 440 g, 450 g, 460 g, 470 g, 480 g, 490 g, 500 g, 510 g, 520 g, 530 g, 540 g, 550 g, 560 g, 570 g, 580 g, 590 g, 600 g, 610 g, 620 g, 630 g, 640 g, 650 g, 660 g, 670 g, 680 g, 690 g, 700 g, 710 g, 720 g, 730 g, 740 g, 750 g, 760 g, 770 g, 780 g, 790 g, 800 g, 810 g, 820 g, 830 g, 840 g, 850 g, 860 g, 870 g, 880 g, 890 g, 900 g, 910 g, 920 g, 930 g, 940 g, 950 g, 960 g, 970 g, 980 g, 990 g, 1000 g, 1100 g, 1200 g, 1300 g, 1400 g, 1500 g, 1600 g, 1700 g, 1800 g, 1900 g, 2000 g, 2100 g, 2200 g, 2300 g, 2400 g, 2500 g, 2600 g, 2700 g, 2800 g, 2900 g, 3000 g, 3250 g, 3500 g, 3750 g, 4000 g, 4250 g, 4500 g, 4750 g, 5000 g, 5500 g, 6000 g, 6500 g, 7000 g, 7500 g, 8000 g, 8500 g, 9000 g, 9500 g, 10000 g, or a number or a range between any two of these values. The centrifugation steps can comprise a period of time of about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 110, 120, 128, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, or a number or a range between any two of these values, minutes. The centrifuging can performed for about 10 minutes to about 20 minutes. Centrifuging the supernatant at a second centrifugal force can comprise centrifuging at 8000 g for about 20 minutes. Recovering the first organelle complexes from the second solution can comprise tangential flow filtration (TFF). TFF can be performed with a low viscosity buffer, and in some embodiments said low viscosity buffer reduces the shear rate. The viscosity of the TFF buffer can be, can be about, can be at least, or can be at most, 1 centipoise (cP), 2 cP, 3 cP, 4 cP, 5 cP, 6 cP, 7 cP, 8 cP, 9 cP, 10 cP, 11 cP, 12 cP, 13 cP, 14 cP, 15 cP, 16 cP, 17 cP, 18 cP, 19 cP, 20 cP, 21 cP, 22 cP, 23 cP, 24 cP, 25 cP, 26 cP, 27 cP, 28 cP, 29 cP, 30 cP, 31 cP, 32 cP, 33 cP, 34 cP, 35 cP, 36 cP, 37 cP, 38 cP, 39 cP, 40 cP, 41 cP, 42 cP, 43 cP, 44 cP, 45 cP, 46 cP, 47 cP, 48 cP, 49 cP, 50 cP, 51 cP, 52 cP, 53 cP, 54 cP, 55 cP, 56 cP, 57 cP, 58 cP, 59 cP, 60 cP, 61 cP, 62 cP, 63 cP, 64 cP, 65 cP, 66 cP, 67 cP, 68 cP, 69 cP, 70 cP, 71 cP, 72 cP, 73 cP, 74 cP, 75 cP, 76 cP, 77 cP, 78 cP, 79 cP, 80 cP, 81 cP, 82 cP, 83 cP, 84 cP, 85 cP, 86 cP, 87 cP, 88 cP, 89 cP, 90 cP, 91 cP, 92 cP, 93 cP, 94 cP, 95 cP, 96 cP, 97 cP, 98 cP, 99 cP, 100 cP, 200 cP, 300 cP, 400 cP, 500 cP, 600 cP, 700 cP, 800 cP, 900 cP, 1000 cP, 2500 cP, 5000 cP, 7500 cP, 10000 cP, or a number or a range between any two of these values. The temperature at which TFF is performed can be, can be about, can be at least, or can be at most, 0° C., 1° C., 2° C., 3° C., 4° C., 5° C., 6° C., 7° C., 8° C., 9° C., 10° C., 11° C., 12° C., 13° C., 14° C., 15° C., 16° C., 17° C., 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., or a number or a range between any two of these values. The shear rate of the TFF procedure and/or flow device (e.g., reducer flow device) can be, can be about, can be at least, or can be at most, 1 sec⁻¹, 2 sec⁻¹, 3 sec⁻¹, 4 sec⁻¹, 5 sec⁻¹, 6 sec⁻¹, 7 sec⁻¹, 8 sec⁻¹, 9 sec⁻¹, 10 sec⁻¹, 11 sec⁻¹, 12 sec⁻¹, 13 sec⁻¹, 14 sec⁻¹, 15 sec⁻¹, 16 sec⁻¹, 17 sec⁻¹, 18 sec⁻¹, 19 sec⁻¹, 20 sec⁻¹, 25 sec⁻¹, 30 sec⁻¹, 35 sec⁻¹, 40 sec⁻¹, 45 sec⁻¹, 50 sec⁻¹, 60 sec⁻¹, 70 sec⁻¹, 80 sec⁻¹, 90 sec⁻¹, 100 sec⁻¹, 110 sec⁻¹, 120 sec⁻¹, 128 sec⁻¹, 130 sec⁻¹, 140 sec⁻¹, 150 sec⁻¹, 160 sec⁻¹, 170 sec⁻¹, 180 sec⁻¹, 190 sec⁻¹, 200 sec⁻¹, 210 sec⁻¹, 220 sec⁻¹, 230 sec⁻¹, 240 sec⁻¹, 250 sec⁻¹, 260 sec⁻¹, 270 sec⁻¹, 280 sec⁻¹, 290 sec⁻¹, 300 sec⁻¹, 310 sec⁻¹, 320 sec⁻¹, 330 sec⁻¹, 340 sec⁻¹, 350 sec⁻¹, 360 sec⁻¹, 370 sec⁻¹, 380 sec⁻¹, 390 sec⁻¹, 400 sec⁻¹, 410 sec⁻¹, 420 sec⁻¹, 430 sec⁻¹, 440 sec⁻¹, 450 sec⁻¹, 460 sec⁻¹, 470 sec⁻¹, 480 sec⁻¹, 490 sec⁻¹, 500 sec⁻¹, 510 sec⁻¹, 520 sec⁻¹, 530 sec⁻¹, 540 sec⁻¹, 550 sec⁻¹, 560 sec⁻¹, 570 sec⁻¹, 580 sec⁻¹, 590 sec⁻¹, 600 sec⁻¹, 610 sec⁻¹, 620 sec⁻¹, 630 sec⁻¹, 640 sec⁻¹, 650 sec⁻¹, 660 sec⁻¹, 670 sec⁻¹, 680 sec⁻¹, 690 sec⁻¹, 700 sec⁻¹, 710 sec⁻¹, 720 sec⁻¹, 730 sec⁻¹, 740 sec⁻¹, 750 sec⁻¹, 760 sec⁻¹, 770 sec⁻¹, 780 sec⁻¹, 790 sec⁻¹, 800 sec⁻¹, 810 sec⁻¹, 820 sec⁻¹, 830 sec⁻¹, 840 sec⁻¹, 850 sec⁻¹, 860 sec⁻¹, 870 sec⁻¹, 880 sec⁻¹, 890 sec⁻¹, 900 sec⁻¹, 910 sec⁻¹, 920 sec⁻¹, 930 sec⁻¹, 940 sec⁻¹, 950 sec⁻¹, 960 sec⁻¹, 970 sec⁻¹, 980 sec⁻¹, 990 sec⁻¹, 1000 sec⁻¹, 1100 sec⁻¹, 1200 sec⁻¹, 1300 sec⁻¹, 1400 sec⁻¹, 1500 sec⁻¹, 1600 sec⁻¹, 1700 sec⁻¹, 1800 sec⁻¹, 1900 sec⁻¹, 2000 sec⁻¹, 2100 sec⁻¹, 2200 sec⁻¹, 2300 sec⁻¹, 2400 sec⁻¹, 2500 sec⁻¹, 2600 sec⁻¹, 2700 sec⁻¹, 2800 sec⁻¹, 2900 sec⁻¹, 3000 sec⁻¹, 3250 sec⁻¹, 3500 sec⁻¹, 3750 sec⁻¹, 4000 sec⁻¹, 4250 sec⁻¹, 4500 sec⁻¹, 4750 sec⁻¹, 5000 sec⁻¹, 5500 sec⁻¹, 6000 sec⁻¹, 6500 sec⁻¹, 7000 sec⁻¹, 7500 sec⁻¹, 8000 sec⁻¹, 8500 sec⁻¹, 9000 sec⁻¹, 9500 sec⁻¹, 10000 sec⁻¹, or a number or a range between any two of these values. TFF can be performed with a buffer comprising human albumin (HA). Recovering the first organelle complexes from the second solution can comprise TFF performed using a TFF membrane. The molecular weight cutoff of the TFF membrane can be, can be about, can be at least, or can be at most, 10 kDa, 11 kDa, 12 kDa, 13 kDa, 14 kDa, 15 kDa, 16 kDa, 17 kDa, 18 kDa, 19 kDa, 20 kDa, 25 kDa, 30 kDa, 35 kDa, 40 kDa, 45 kDa, 50 kDa, 60 kDa, 70 kDa, 80 kDa, 90 kDa, 100 kDa, 110 kDa, 120 kDa, 128 kDa, 130 kDa, 140 kDa, 150 kDa, 160 kDa, 170 kDa, 180 kDa, 190 kDa, 200 kDa, 210 kDa, 220 kDa, 230 kDa, 240 kDa, 250 kDa, 260 kDa, 270 kDa, 280 kDa, 290 kDa, 300 kDa, 310 kDa, 320 kDa, 330 kDa, 340 kDa, 350 kDa, 360 kDa, 370 kDa, 380 kDa, 390 kDa, 400 kDa, 410 kDa, 420 kDa, 430 kDa, 440 kDa, 450 kDa, 460 kDa, 470 kDa, 480 kDa, 490 kDa, 500 kDa, 510 kDa, 520 kDa, 530 kDa, 540 kDa, 550 kDa, 560 kDa, 570 kDa, 580 kDa, 590 kDa, 600 kDa, 610 kDa, 620 kDa, 630 kDa, 640 kDa, 650 kDa, 660 kDa, 670 kDa, 680 kDa, 690 kDa, 700 kDa, 710 kDa, 720 kDa, 730 kDa, 740 kDa, 750 kDa, 760 kDa, 770 kDa, 780 kDa, 790 kDa, 800 kDa, 810 kDa, 820 kDa, 830 kDa, 840 kDa, 850 kDa, 860 kDa, 870 kDa, 880 kDa, 890 kDa, 900 kDa, 910 kDa, 920 kDa, 930 kDa, 940 kDa, 950 kDa, 960 kDa, 970 kDa, 980 kDa, 990 kDa, 1000 kDa, 1100 kDa, 1200 kDa, 1300 kDa, 1400 kDa, 1500 kDa, 1600 kDa, 1700 kDa, 1800 kDa, 1900 kDa, 2000 kDa, 2100 kDa, 2200 kDa, 2300 kDa, 2400 kDa, 2500 kDa, 2600 kDa, 2700 kDa, 2800 kDa, 2900 kDa, 3000 kDa, 3250 kDa, 3500 kDa, 3750 kDa, 4000 kDa, 4250 kDa, 4500 kDa, 4750 kDa, 5000 kDa, 5500 kDa, 6000 kDa, 6500 kDa, 7000 kDa, 7500 kDa, 8000 kDa, 8500 kDa, 9000 kDa, 9500 kDa, 10000 kDa, or a number or a range between any two of these values.

The method further can comprise freezing the organelle complexes. Freezing can be performed by mildly suspending the organelle complexes in a buffer for freezing (e.g., a preservation buffer). The buffer for freezing may be a buffer employed in the first solution, but not including a surfactant, and may further comprise a cryoprotectant. Said cryoprotectant can comprise human albumin (HA) and/or glycerol. The percentage of glycerol can be, can be about, can be at least, or can be at most, 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, or a number or a range between any two of these values. The percentage of HA can be, can be about, can be at least, or can be at most, 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.00%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 60%, 70%, 80%, 90%, 100%, or a number or a range between any two of these values. Exemplary cryoprotectants are known in the art and include, for example, glycerol, sucrose, trehalose, dimethyl sulfoxide (DMSO), ethylene glycol, propylene glycol, diethyl glycol, triethylene glycol, glycerol-3-phosphate, proline, sorbitol, formamide, and polymers. Thus, the organelle complexes provided herein can be stored by freezing. In the method of the present disclosure, organelle complexes may not be frozen if cryopreservation is not necessary, e.g., the organelle complexes may be used when freshly isolated. In other embodiments, the organelle complexes may be stored at 4° C.±3° C. or on ice. In some embodiments, the organelle complexes provided herein produced by the method provided herein may be stored in liquid nitrogen, at about −80° C.±3° C. or lower, about −20° C.±3° C. or lower, or about 4° C.±3° C. In some embodiments, the methods further comprise thawing the organelle complexes after freezing. In some embodiments, the methods for thawing the organelle complexes comprise rapidly thawing the organelle complexes, for example, within about 5 minutes or within about 1 minute. In some embodiments, the organelle complexes are thawed in a warm bath having a temperature of about 20° C.±3° C. to about 37° C.±3° C. In some embodiments, the organelle complexes are thawed at a temperature of about 20° C.±3° C. or colder. In some embodiments, the organelle complexes may be stored for days, weeks, or months, or longer, and retain the capacity to function after thawing.

In some embodiments, the methods provided herein do not comprise homogenization; In some embodiments, the methods comprise homogenization but the homogenization is carried only to the extent that it does not cause any bubbles or bubbles to the solution relative to the cell or tissue. In some embodiments, the methods also do not comprise freeze-thawing of cells. In some embodiments, the methods of this disclosure do not require one or more filtration steps in purifying organelle complexes recovered from cells. In some embodiments, the method does not comprise the application of shear forces (e.g., douncing, passing through a needle) and/or the addition of proteases to the cells, the first solution and/or the second solution. In some embodiments, the methods of the present disclosure do not include other methods of disrupting the cell membrane (e.g., sonication, treatment with a strong stream of water to the extent that a solution produces bubbles, or to the extent that the solution foams) during the whole process of collecting first organelle complexes from cells. In some embodiments, the method of the present disclosure is performed without performing any processes that may substantially cause physical, chemical, or physiological damage to the organelle complexes, although a freeze-thaw cycle can be applied to the organelle complexes for storage. Thus, the methods of this disclosure are capable of obtaining organelle complexes with minimal damage.

In some embodiments, the methods provided herein comprise encapsulating the first organelle complexes in lipid membrane-based vesicles. There is provided, in some embodiments, a method for producing lipid membrane-based vesicles encapsulating organelle complexes or a population thereof or a composition comprising the vesicles, comprising bringing an aqueous solution comprising organelle complexes and an organic phase (for example, ethanol solution) comprising a lipid that can form lipid membrane into contact with each other in the confluent channel within a micro flow channel device to mix them. Methods of encapsulating in lipid membrane-based vesicles are disclosed in PCT Patent Application Publication No. WO2021/132735, the contents of which are incorporated herein by reference in its entirety.

### Methods of Treatment

There are provided, in some embodiments, methods for treating or preventing a disease or disorder. In some embodiments, the method comprises: contacting cells of a subject in need thereof with an effective amount of: (i) an organelle complexes population provided herein; (ii) a composition provided herein (e.g., a composition comprising an organelle complexes population); and/or (iii) a formulation provided herein, thereby treating or preventing the disease or disorder.

There are provided, in some embodiments, methods for treating or preventing a disease or disorder associated with mitochondrial dysfunction. In some embodiments, the method comprises: contacting cells of a subject in need thereof with an effective amount of: (i) an organelle complexes population provided herein; (ii) a composition provided herein (e.g., a composition comprising an organelle complexes population); and/or (iii) a formulation provided herein, thereby treating or preventing a disease or disorder associated with mitochondrial dysfunction.

The present disclosure also provides use of organelle complexes population in the manufacture of a medicament for treating the diseases and disorders provided herein. In some embodiments, the organelle complexes population is administered to the subject in combination with one or more additional agents and/or additional therapies designed to treat the disease or disorder. In some embodiments, the present disclosure provides methods for treating diseases and disorders associated with mitochondrial dysfunction or diseases or disorders that otherwise benefit from the supplementation of healthy, functional mitochondria.

Contacting cells of the subject can comprise a route of administration selected from the group comprising intravenous administration, intra-arterial administration, intra-tracheal administration, subcutaneous administration, intramuscular administration, inhalation, intrapulmonary administration, and intra-ocular administration. The organelle complexes population can be administered locally or systemically.

The wording “local administration” or “topic administration” as used herein indicates any route of administration by which an organelle complexes population is brought in contact with the body of the individual, so that the resulting organelle complexes population location in the body is topic (limited to a specific tissue, organ or other body part where the imaging is desired). Exemplary local administration routes include injection into a particular tissue by a needle, gavage into the gastrointestinal tract, and spreading a solution containing organelle complexes population on a skin surface.

The wording “systemic administration” as used herein indicates any route of administration by which an organelle complexes population is brought in contact with the body of the individual, so that the resulting organelle complexes population location in the body is systemic (i.e. non limited to a specific tissue, organ or other body part where the imaging is desired). Systemic administration includes enteral and parenteral administration. Enteral administration is a systemic route of administration where the substance is given via the digestive tract, and includes but is not limited to oral administration, administration by gastric feeding tube, administration by duodenal feeding tube, gastrostomy, enteral nutrition, and rectal administration. Parenteral administration is a systemic route of administration where the substance is given by route other than the digestive tract and includes but is not limited to intravenous administration, intra-arterial administration, intramuscular administration, subcutaneous administration, intradermal, administration, intraperitoneal administration, and intravesical infusion.

In another aspect, this disclosure provides pharmaceutically acceptable compositions which comprise a therapeutically-effective amount of an organelle complexes population disclosed herein. As described in detail below, the pharmaceutical compositions of this disclosure may be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension: (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the organelle complexes population. The pharmaceutical compositions can comprise one or more pharmaceutically-acceptable carriers. The phrase “therapeutically-effective amount” as used herein can refer to that amount of an organelle complexes population disclosed herein which is effective for producing some desired therapeutic effect, e.g., cancer treatment, at a reasonable benefit/risk ratio.

The phrase “pharmaceutically acceptable” is employed herein to refer to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically-acceptable carrier” as used herein means a pharmaceutically-acceptable material, composition or vehicle, such as a liquid or solid filler, diluent, excipient, solvent or encapsulating material, involved in carrying or transporting the subject chemical from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth: (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (1) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

Formulations useful in the methods of this disclosure include those suitable for oral, nasal, topical (including buccal and sublingual), rectal, vaginal, aerosol and/or parenteral administration. The formulations may conveniently be presented in unit dosage form and may be prepared by any methods well known in the art of pharmacy. The amount of active ingredient (e.g., organelle complexes population) which can be combined with a carrier material to produce a single dosage form will vary depending upon the host being treated, the particular mode of administration. The amount of active ingredient, which can be combined with a carrier material to produce a single dosage form will generally be that amount of the organelle complexes population which produces a therapeutic effect. Generally, out of one hundred percent, this amount will range from about 1% to about 99% of active ingredient, preferably from about 5% to about 70%, most preferably from about 10% to about 30%.

Suspensions, in addition to the active agent may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Dosage forms for the topical or transdermal administration of organelle complexes population include powders, sprays, ointments, pastes, creams, lotions, gels, solutions, patches and inhalants. The active component may be mixed under sterile conditions with a pharmaceutically-acceptable carrier, and with any preservatives, buffers, or propellants which may be required.

The ointments, pastes, creams and gels may contain excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Ophthalmic formulations, eye ointments, powders, solutions and the like, are also contemplated as being within the scope of this disclosure.

Examples of suitable aqueous and nonaqueous carriers which may be employed in the pharmaceutical compositions of this disclosure include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Actual dosage levels of the active ingredients in the pharmaceutical compositions of this disclosure may be determined by the methods of this disclosure so as to obtain an amount of the active ingredient, which is effective to achieve the desired therapeutic response for a particular subject, composition, and mode of administration, without being toxic to the subject.

The disease or disorder can be selected from the group consisting of diabetes (Type I and Type II), metabolic disease, ocular disorders associated with mitochondrial dysfunction, hearing loss, mitochondrial toxicity associated with therapeutic agents, mitochondrial dysfunction associated with Space travel, cardiotoxicity associated with chemotherapy or other therapeutic agents, a mitochondrial dysfunction disorder, and migraine.

The disease or disorder can be selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.

The disease or disorder can be an ischemia-related disease or disorder, a genetic disorder, an aging disease or disorder, a neurodegenerative condition, a cardiovascular condition, a cancer, an autoimmune disease, an inflammatory disease, a fibrotic disorder, or any combination thereof. The ischemia-related disease or disorder can be selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ. The neurodegenerative condition can be selected from the group consisting of dementia, Friedrich's ataxia, amyotrophic lateral sclerosis, mitochondrial encephalopathy, lactic acidosis, and stroke-like episodes (MELAS), myoclonic epilepsy with ragged red fibers (MERFF), epilepsy, Parkinson's disease, Alzheimer's disease, or Huntington's Disease. Exemplary neuropsychiatric disorders include bipolar disorder, schizophrenia, depression, addiction disorders, anxiety disorders, attention deficit disorders, personality disorders, autism, and Asperger's disease. The cardiovascular condition can be selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism. The disease or disorder can be acute respiratory distress syndrome (ARDS) or pre-eclampsia or intrauterine growth restriction (IUGR).

Exemplary ischemia-related diseases and disorders include cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing (e.g., an acute wound or a chronic wound; a cut, laceration, compression wound, burn wound (e.g., chemical, heat or flame, wind, or sun burn), or a wound resulting from a medical or surgical intervention), spinal cord injury, sickle cell disease, and reperfusion injury of a transplanted organ. In some embodiments, the organelle complexes population may treat, prevent, ameliorate, and/or improve clinical condition due to ischemia-reperfusion injury. In some embodiments, the organelle complexes population may improve Ejection Fraction (EF), inhibit cardiac hypertrophy, and/or treat, prevent, ameliorate, and/or improve fibrosis after ischemia-reperfusion injury.

Also provided herein are kits comprising one or more compositions (e.g., a formulation comprising an organelle complexes population) described herein, in suitable packaging, and may further comprise written material that can include instructions for use, discussion of clinical studies, listing of side effects, and the like. Such kits may also include information, such as scientific literature references, package insert materials, clinical trial results, and/or summaries of these and the like, which indicate or establish the activities and/or advantages of the composition, and/or which describe dosing, administration, side effects, drug interactions, or other information useful to the health care provider. Such information may be based on the results of various studies, for example, studies using experimental animals involving in vivo models and studies based on human clinical trials. A kit may comprise one or more unit doses described herein.

### EXAMPLES

Some aspects of the embodiments discussed above are disclosed in further detail in the following examples, which are not in any way intended to limit the scope of the present disclosure.

### Example 1

### Characterization of Organelle Complexes Populations

The composition of the organelle complexes populations provided herein, as well as the functional differences between them (and homogenized mitochondria obtained via currently available methods), was examined in a series of experiments.

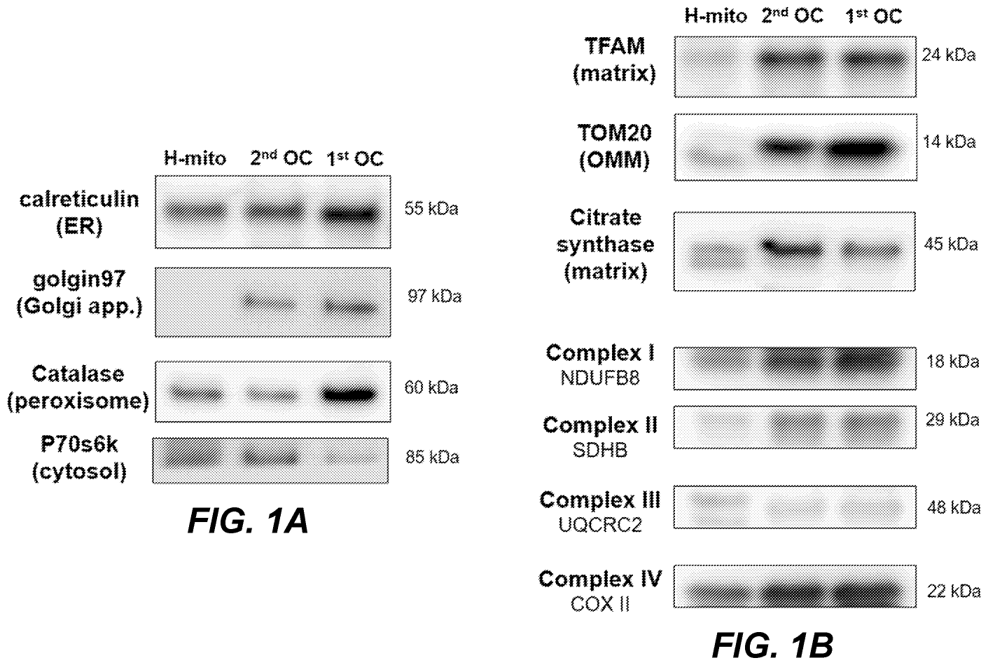

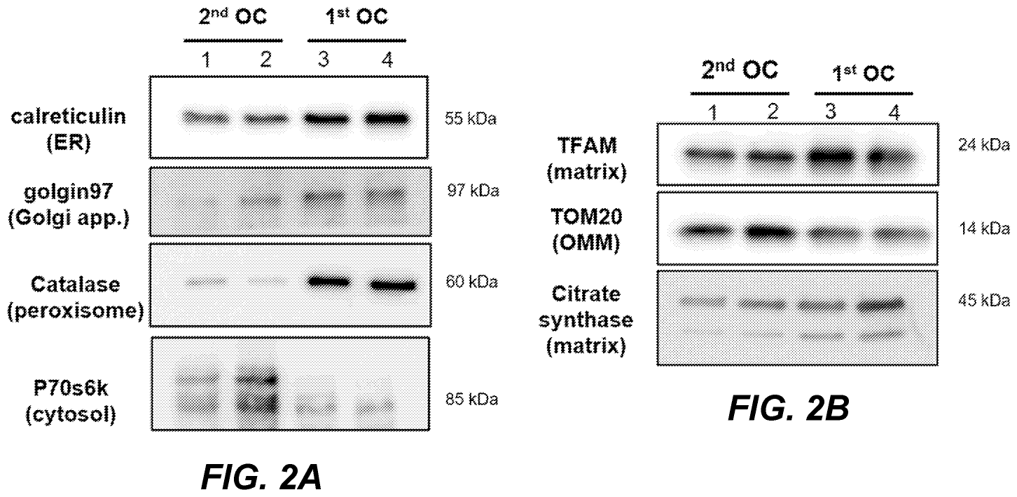

Homogenized mitochondria, first organelle complexes, and second organelle complexes were isolated from HeLa cells (Pierce μg/mL: HeLa H-mito, 650.1; HeLa 2^{nd} OC, 238.3; HeLa 1^{st} OC, 660). Isolated homogenized mitochondria and organelle complexes populations were resuspended in H₂O (Milli-Q) and Laemmli sample buffer. The final concentration was adjusted to ˜3 mg in 15 ml and samples were then boiled at 96° C. for 5 min. The samples were ran on 4-20% gradient gels (100V, 75 min). The antibodies used were as follows: AMPKa (D5A2) Rabbit mAb #5831; Phospho-AMPKα (Thrl72) (40H9) Rabbit mAb #2535; TFAM (D5C8) Rabbit mAb #8076; Tom20 (F-10) Mouse mAb #sc-17764; p70 S6 Kinase (49D7) Rabbit mAb #2708; Golgin-97 (D8P2K) Rabbit mAb #13192; Catalase (D4P7B) XP® Rabbit mAb #12980; Citrate Synthase (D7V8B) Rabbit mAb #14309; Calreticulin (D3E6) XP® Rabbit mAb #12238; Anti-Lamin A/C Antibody (E-1) #sc-376248; and OxPhos Human WB Antibody Cocktail, Rabbit mAb #45-8199. FIGS. 1A-1B depict non-limiting exemplary data related to characterization of the organelle complexes populations provided herein. FIG. 1A depicts intracellular structures/organelles western blot protein analysis of homogenized mitochondria (H-mito), first organelle complexes (1^{st} OC), and second organelle complexes (2^{nd} OC) prepared using the methods disclosed herein from HeLa cells. FIG. 1B depicts mitochondria markers western blot protein analysis of homogenized mitochondria (H-mito), first organelle complexes (1^{st} OC), and second organelle complexes (2^{nd} OC) prepared using the methods disclosed herein from HeLa cells. FIGS. 2A-2B depict non-limiting exemplary data related to characterization of the organelle complexes populations provided herein. First organelle complexes and second organelle complexes were isolated from HEK293T cells. FIG. 2A depicts intracellular structures/organelles western blot protein analysis of first organelle complexes (1^{st} OC) and second organelle complexes (2nd OC) prepared using the methods disclosed herein from HEK293T cells. FIG. 2B depicts mitochondria markers western blot protein analysis of first organelle complexes (1^{st} OC) and second organelle complexes (2^{nd} OC) prepared using the methods disclosed herein from HEK293T cells. These data show that organelle complexes populations isolated from different types of cells show similar results with regards to differences between first organelle complexes and second organelle complexes. Moreover, these data show a large difference in mitochondrial markers between first organelle complexes and second organelle complexes as compared to homogenized mitochondria.

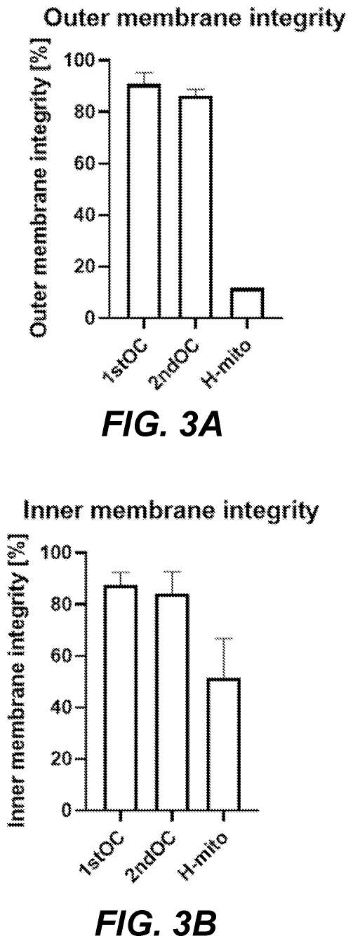

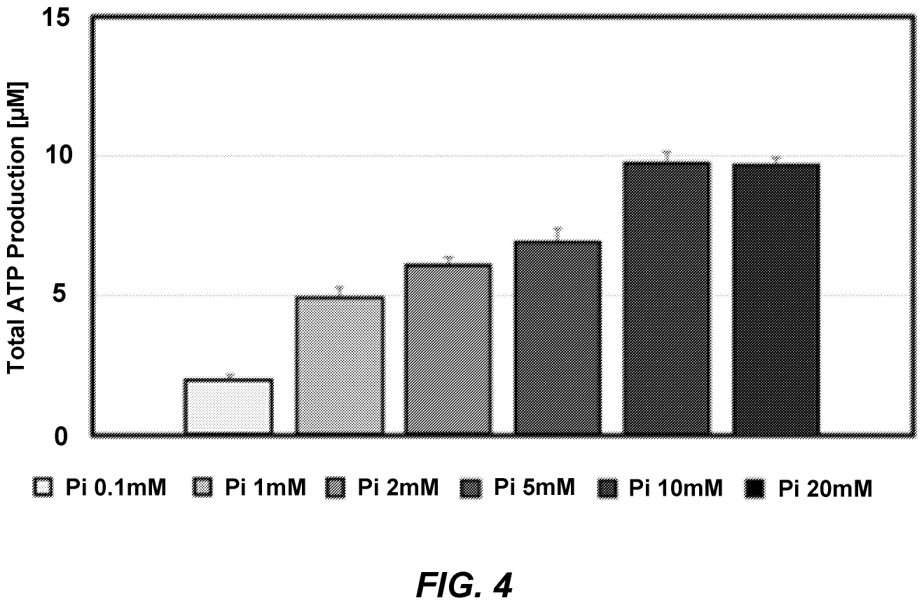

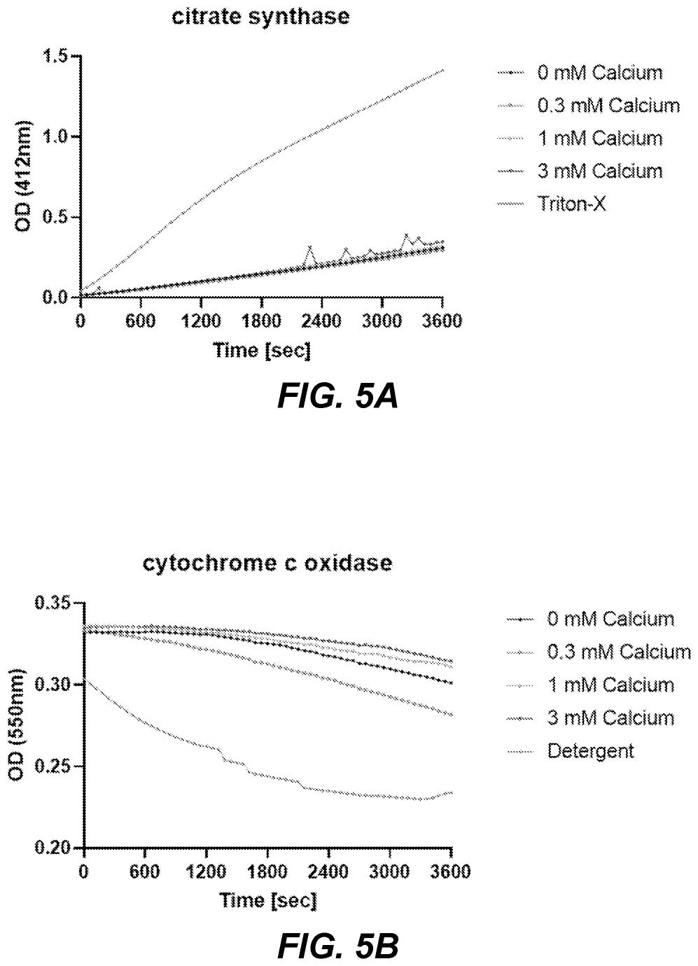

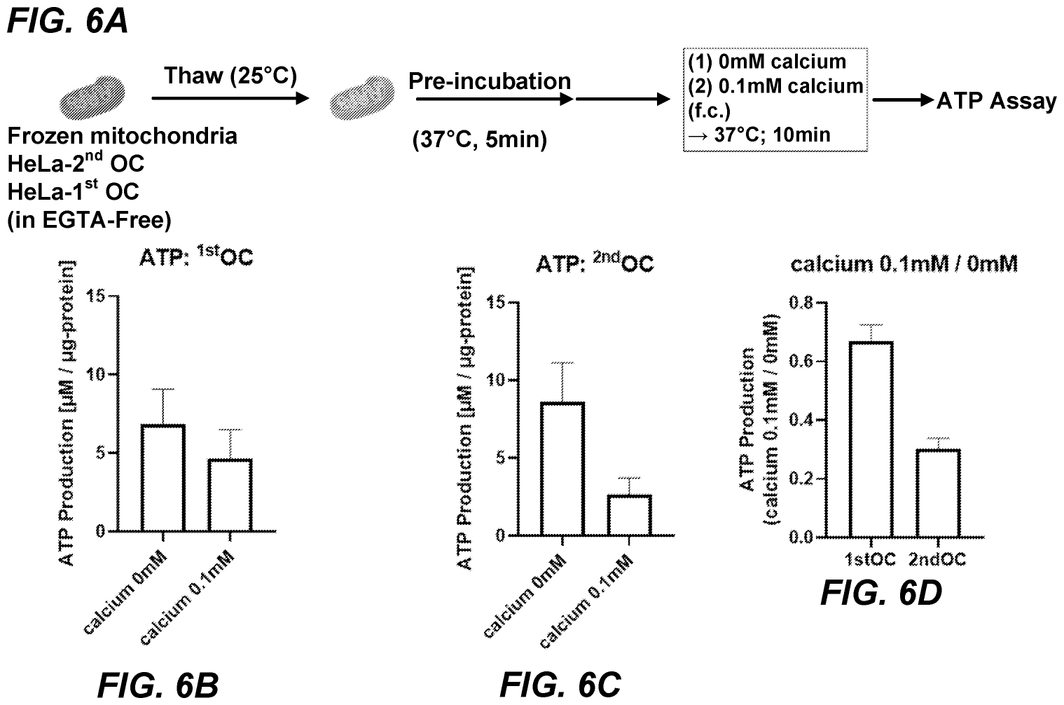

FIGS. 3A-3B depict non-limiting exemplary data related to the structural integrity of first organelle complexes and second organelle complexes: outer membrane integrity (FIG. 3A), inner membrane integrity (FIG. 3B). In some embodiments, membrane integrity (%)=((amount of end−product with detergent−amount of end-product without detergent)/amount of end-product with detergent). FIG. 4 depicts non-limiting exemplary data related to the production of ATP by a first organelle complexes population at a range of Pi concentrations. FIGS. 5A-5B depict non-limiting exemplary data related to first organelle complexes structural integrity. Citrate synthase activity (FIG. 5A) and cytochrome c oxidase activity (FIG. 5B) of first organelle complexes were assayed at a range of Ca²⁺ concentrations. FIG. 6A depicts the experimental setup and FIGS. 6B-6D depict data related to the structural integrity of first organelle complexes and second organelle complexes. FIGS. 6B-6D depict ATP production of second organelle complexes (2^{nd} OC; FIG. 6C) and first organelle complexes (1^{st} OC; FIG. 6B) at the indicated concentrations, and FIG. 6D depicts the ATP production ratio of both organelle complexes. Collectively, these data demonstrate the structural and functional differences between the organelle complexes populations provided herein and homogenized mitochondria generated by currently available methods. The first organelle complexes and second organelle complexes provided herein comprises various organelles (in addition to mitochondria), exhibit advantageous functional properties, and maintain structural integrity in a range of extracellular environments.

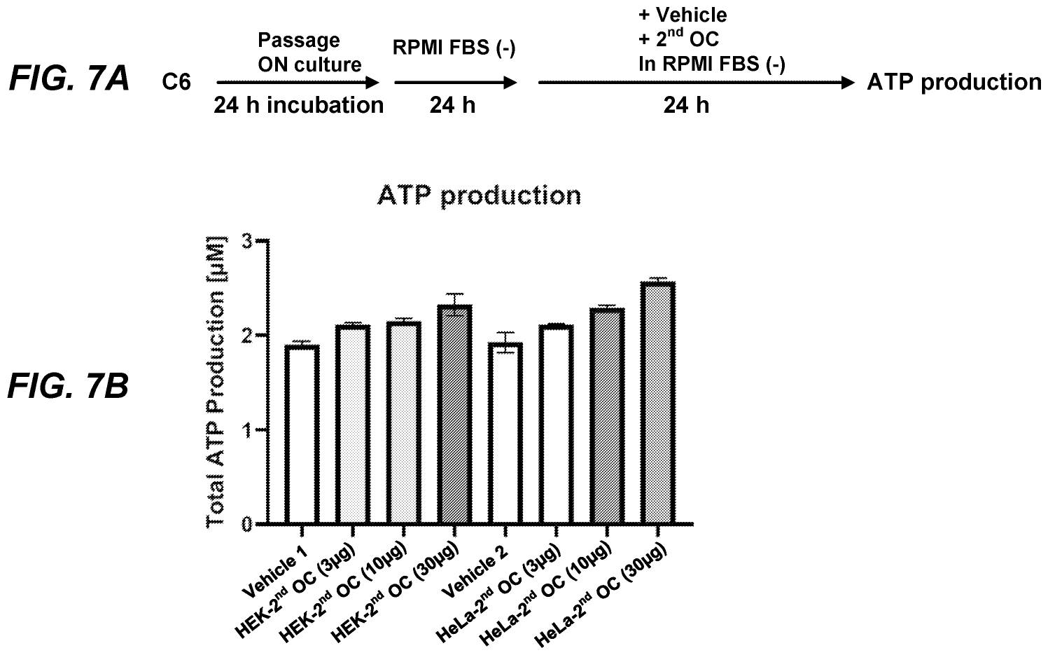

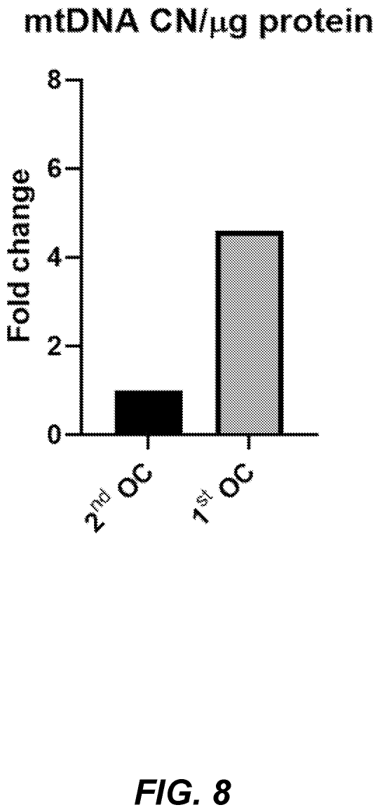

The impact of cellular uptake of second organelle complexes on ATP production was next investigated. FIGS. 7A-7B depict the experimental setup (FIG. 7A) and data (FIG. 7B) related to the effect of second organelle complexes (2^{nd} OC) on ATP production. Cellular uptake of second organelle complexes was found to increase ATP production in a dose-dependent manner.

qPCR analysis of the first organelle complexes and second organelle complexes was then performed. FIG. 8 depicts qPCR data related to the first organelle complexes (1^{st} OC) and second organelle complexes (2^{nd} OC). The data demonstrate the large difference in relative mitochondrial DNA copy number (mtDNA CN) between first organelle complexes and second organelle complexes when as quantified by qPCR, with first organelle complexes having between ˜2-6 fold more mtDNA CN in the isolated organelle complex.

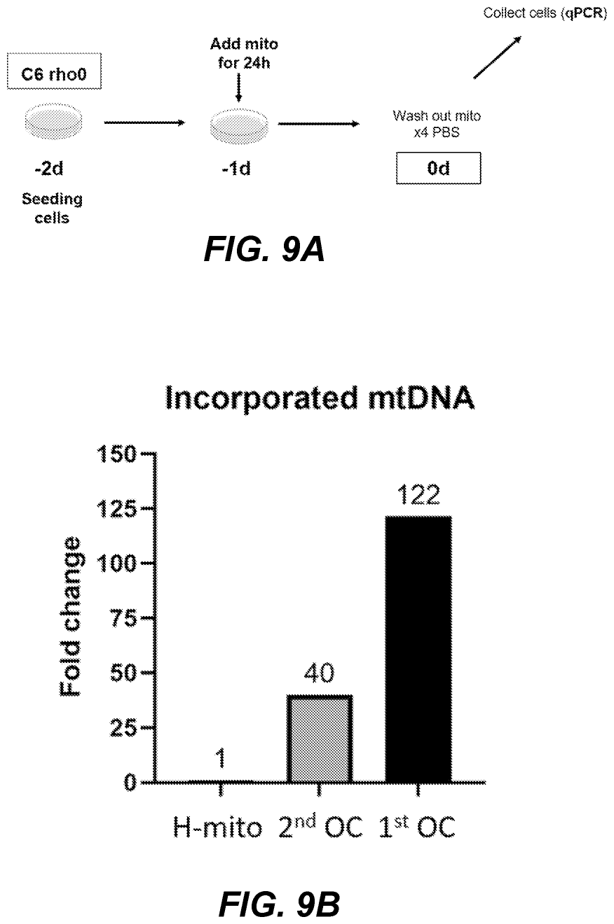

A comparison of incorporation of mtDNA in recipient C6 rho0 cells incubated with homogenized mitochondria, first organelle complexes, and second organelle complexes was next performed. The recipient employed was a C6 rho0 cell line, which is a derivative of the rat C6 glioma cells and have lost their mitochondrial DNA (mtDNA) as a result of transient transfection with plasmid. The mitochondria donor was HeLa, which is an immortal cell line derived from human cervical cancer cells. Homogenized mitochondria (H-mito), first organelle complexes (1^{st} OC), and second organelle complexes (2^{nd} OC) were isolated from HeLa cells. C6 rho0 cells were cultured in 12 well plates at a cell concentration of 2×10⁵ cells/well. Homogenized mitochondria (H-mito), first organelle complexes (1^{st} OC), and second organelle complexes (2^{nd}OC) were co-incubated at 42 g/well. FIG. 9A depicts the experimental setup and FIG. 9B depicts data related to incorporation of mtDNA in recipient C6 rho0 cells incubated with homogenized mitochondria (H-mito), first organelle complexes (1^{st} OC), and second organelle complexes (2^{nd}OC). FIG. 9B depicts the ratio of incorporated mtDNA of 1^{st} OC and 2^{nd} OC relative to homogenized mitochondria 24 hours after co-incubation with homogenized mitochondria, first organelle complexes, and second organelle complexes. Increased incorporation of mtDNA in the first organelle complexes treated group in recipient C6 rho0 cells was observed. Specifically, a three-fold increase in incorporated mtDNA was observed with first organelle complexes used as a donor.

### Example 2

### Alternate Final Centrifugation Conditions

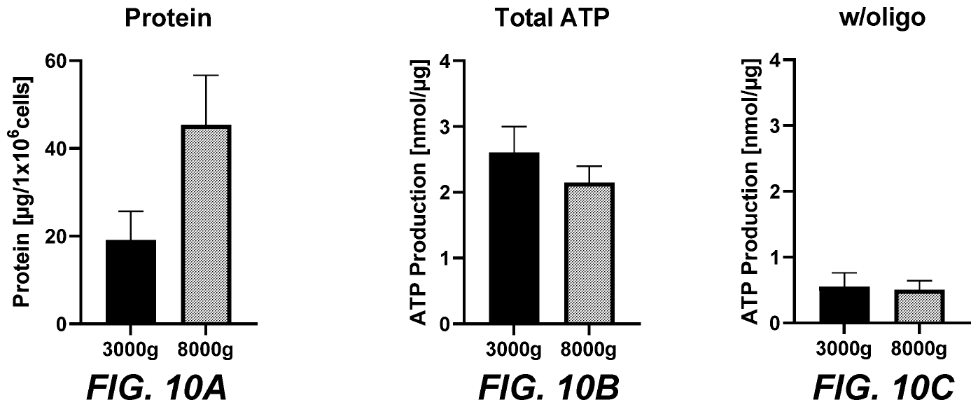

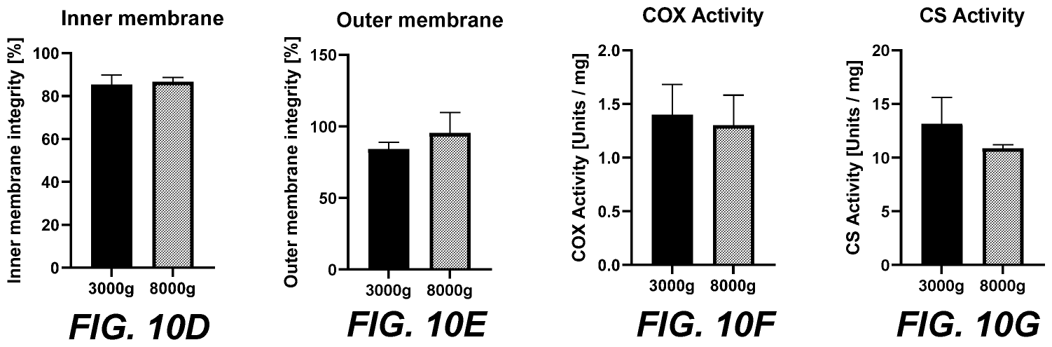

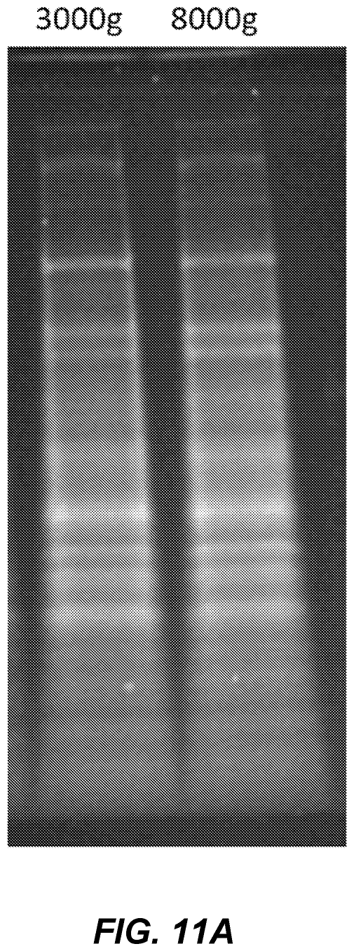

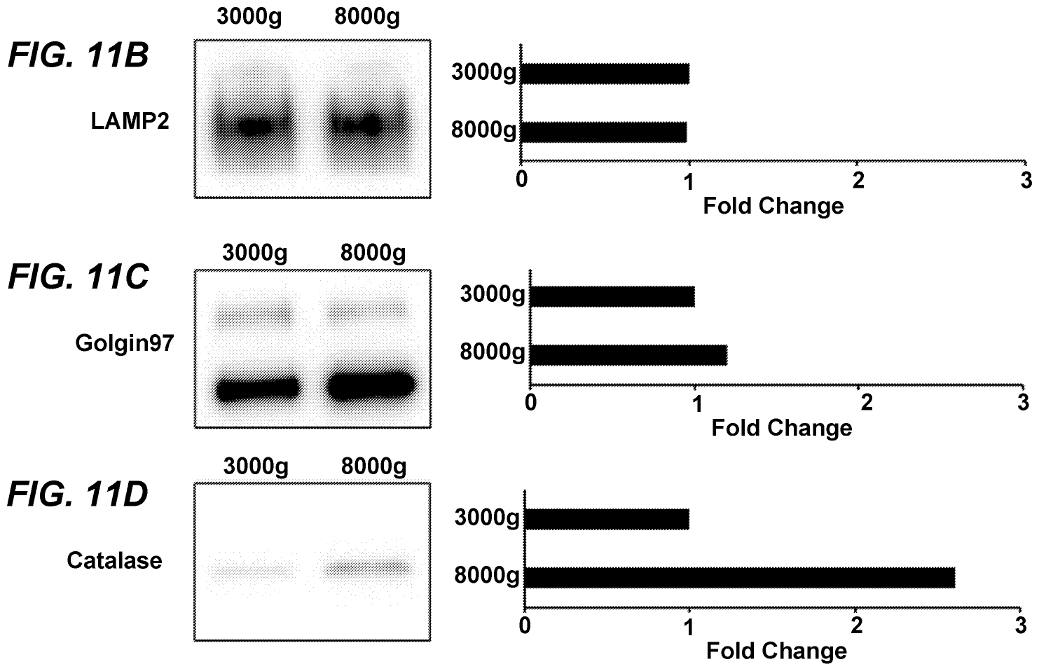

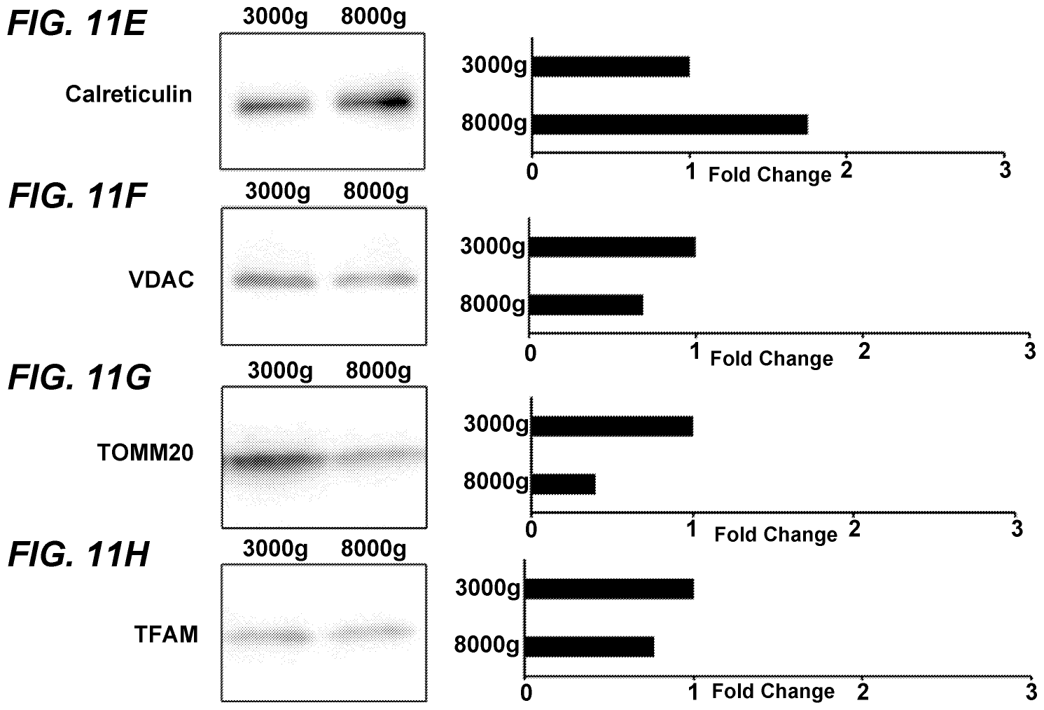

In some embodiments of the methods provided herein, the second solution is centrifuged at a first centrifugal force, the supernatant is collected and centrifuged at a second centrifugal force, and the pellet is collected to recover the first organelle complexes. The consequences increasing the second centrifugal force from 3000 g to 8000 g (with angle, soft brake) and increasing the second centrifugation time from 10 min to 20 min were examined in this Example. FIGS. 10A-10G depict data related to protein concentration (FIG. 10A), total ATP production (FIG. 10B), ATP production in presence of oligomycin (FIG. 10C), inner membrane integrity (FIG. 10D), outer membrane integrity (FIG. 10E), COX activity (FIG. 10F), and citrate synthase (CS) activity (FIG. 10G) of first organelle complexes recovered using final centrifugation conditions of 3000 g (10 min) or 8000 g (20 min). FIGS. 11A-11G depict a stained SDS-PAGE gel (17 μg protein/lane) (FIG. 11A) and data related to protein levels of LAMP2 (Lysosome; FIG. 11B), Golgin97 (Golgi; FIG. 11C), Catalase (Peroxisome; FIG. 11D), Calreticulin (ER; FIG. 11E), VDAC (Mitochondria (Outer); FIG. 11F), TOMM20 (Mitochondria (Outer); FIG. 11G), and TFAM (Mitochondria (matrix); FIG. 11H) of first organelle complexes recovered using final centrifugation conditions of 3000 g (10 min) or 8000 g (20 min).

### Example 3

### Biological Activity of Homogenized Mitochondria, First Organelle Complexes, and Second Organelle Complexes

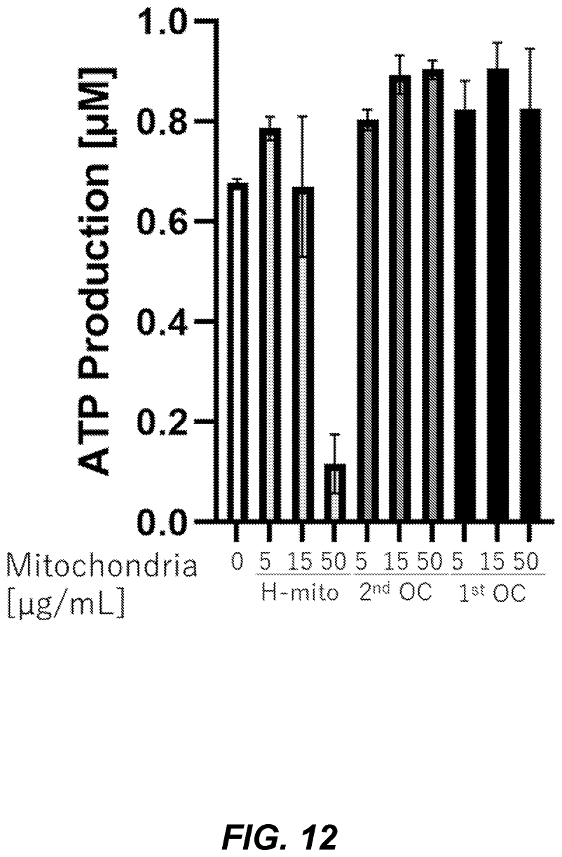

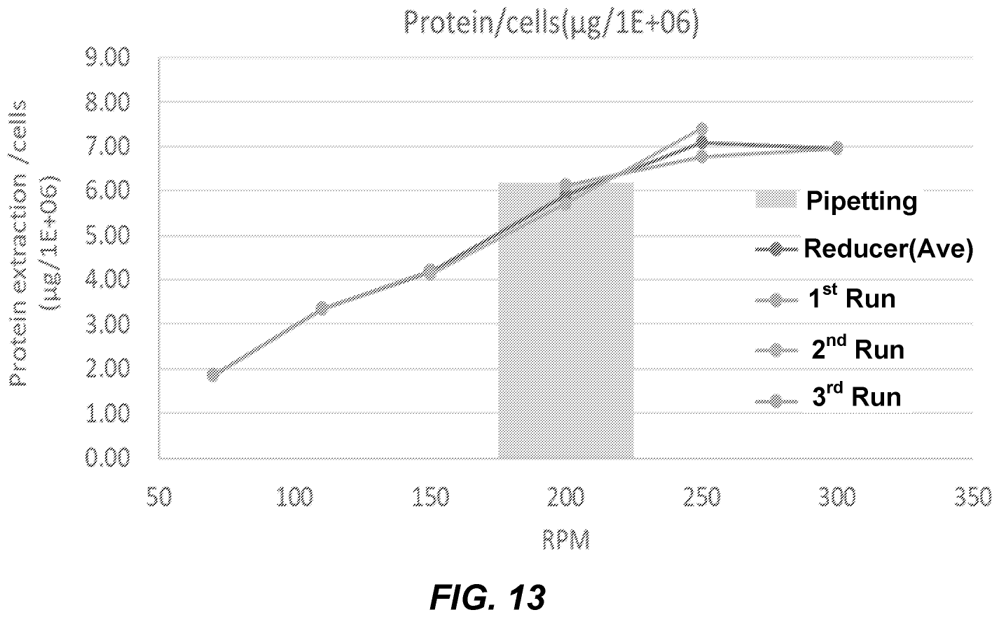

The biological activity of homogenized mitochondria, first organelle complexes, and second organelle complexes was compared in this Example. Fibroblasts (5000 cells/well; 96 well plate) were incubated for approximately 24 hours and then FBS(−) vehicle (Tris-sucrose buffer), homogenized mitochondria, first organelle complexes, or second organelle complexes (each derived from HeLa cells) were added at concentrations of 5 ug/ml, 15 ug/ml, or 50 ug/ml. After a 24 hour incubation ATP production measurements (in triplicate) were performed with Cell Titer-Glo. FIG. 12 depicts data related to the ATP production of fibroblasts incubated with homogenized mitochondria (H-mito), first organelle complexes, or second organelle complexes. These data indicate that high doses of homogenized mitochondria may induce cell death (a trend that was also seen in microscopic observations even at medium doses). Both second organelle complexes and first organelle complexes showed a dose-dependent increase in intracellular ATP production, with first organelle complexes appearing to be more effective at lower doses.

### Example 4

### Reducer Flow Device

As described herein, the first generation method of generating first organelle complexes can be modified and can comprise the use of a flow device (e.g., a reducer flow device) in some embodiments rather than (or in addition to) pipetting (e.g., at Step G) as a means of applying a physical stimulus to the first solution and/or the second solution. FIG. 14 depicts data related to first organelle complexes obtained via a first generation method (employing pipetting as a physical stimulus) versus a modified method employing a reducer flow device as a physical stimulus. It was found that higher protein recovery rate can be obtained using the reducer flow device as compared to the first generation method (comprising pipetting).

### Example 5

### Tangential Flow Filtration

The use of a TFF system during first organelle complexes isolation, including various TFF parameters, such as shear rate and buffer composition, was investigated in this Example. The use of a lower shear rate during TFF purification was first examined. AT the shear rate is approximately 1000-2000 sec⁻¹. It was found that employing a lower shear rate can improve outer membrane integrity in TFF purification (Table 2).

TABLE 2
TFF Shear Rate Testing

| | | | | | | Avg Outer |
|---|---|---|---|---|---|---|
| TFF | Feed flow | | Shear | Running | | membrane |
| batches | rate/min | | rate(sec − 1) | time | | integrity (5) |
| 1 | 150 | ml/min | 2000 | 60 | min | 27 |
| 2 | 70 | ml/min | 1000 | 2.5 | hr | 60 |
| 3 | 50 | ml/min | 700 | 2.5 | hr | 75 |

The effect of buffer viscosity during TFF purification was next examined. The use of buffer with lower viscosity was found improve the outer membrane integrity of first organelle complexes in TFF purification (Table 3). Accordingly, in some embodiments provided herein, a lower viscosity buffer (e.g., mannitol based buffer) is employed for first organelle complexes isolation during TFF.

TABLE 3
TFF Buffer Viscosity

| TFF | | Running | Outer membrane |
|---|---|---|---|
| batches | Buffer | time | integrity % |
| 1 | Tris-sucrose-buffer | 2.5 hr | 60-70 |
| 2 | Tris-mannitol-sucrose | 2.5 hr | 80-90 |
| | buffer | | |

The impact of HA and glycerol in preservation buffers on the outer membrane integrity of mitochondria was next examined (Table 4).

TABLE 4
Preservation Buffer Testing Results

| | Outer | COX Activity |
|---|---|---|
| | membrane | (w/Detergent) |
| Group | Integrity [%] | [Unit/mg] |
| TMSB + 20% GLY + 4% HSA | 97% | 3.7 |
| TMSB + 10% GLY + 4% HSA | 95% | 3.3 |
| TMSB + 10% GLY + 2% HSA | 94% | 3.7 |
| TMSB + 10% GLY + 1% HSA | 94% | 3.7 |
| TMSB + 4% HSA | 87% | 4.0 |
| TMSB + 2% HSA | 86% | 4.3 |
| TMSB + 1% HSA | 88% | 4.2 |
| TMSB (Control) (Buffer only) | 89% | 4.0 |

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An organelle complexes population,
wherein the organelle complexes comprise mitochondria and one or more of endoplasmic reticulum, peroxisomes, lysosomes, and Golgi apparatus,
wherein the organelle complexes are isolated or derived from floating cells and/or frozen cells,
wherein the organelle complexes are depleted of cytosolic macromolecules, and
wherein at least about 80% of the mitochondria of the organelle complexes maintain structural integrity in an extracellular environment.

2. An organelle complexes population,
wherein the organelle complexes comprise mitochondria and one or more of endoplasmic reticulum, peroxisomes, lysosomes, and Golgi apparatus,
wherein the organelle complexes are isolated or derived from cells contacted with a surfactant at a concentration at or above the critical micellar concentration (CMC) for the surfactant,
wherein the organelle complexes are depleted of cytosolic macromolecules, and
wherein at least about 80% of the mitochondria of the organelle complexes maintain structural integrity in an extracellular environment.

3. The organelle complexes population of any one of claims 1-2, wherein structural integrity comprises inner membrane structural integrity and/or outer membrane structural integrity of mitochondria.

4. The organelle complexes population of any one of claims 1-3, wherein at least 2-fold more of the mitochondria of the organelle complexes maintain structural integrity in the extracellular environment as compared to a population of homogenized mitochondria in the extracellular environment.

5. The organelle complexes population of any one of claims 1-4, wherein structural integrity is measured by citrate synthase (CS) activity and/or cytochrome c oxidase (COX) activity.

6. The organelle complexes population of any one of claims 1-5, wherein the extracellular environment comprises a total calcium concentration of about 1 to about 20 mg/dL and/or a free/active calcium concentration of about 1 to about 6 mg/dL.

7. The organelle complexes population of any one of claims 1-6, wherein at least about 80% of the mitochondria of the organelle complexes maintain functional capability.

8. The organelle complexes population of any one of claims 1-7, wherein the mitochondria of the organelle complexes are capable of ATP production.

9. The organelle complexes population of any one of claims 1-8, wherein the organelle complexes population comprise an at least 2-fold to 6-fold greater mitochondrial DNA (mtDNA) copy number as compared to a population of homogenized mitochondria.

10. The organelle complexes population of any one of claims 1-9, wherein the cytosolic macromolecules comprise cytosolic proteins, wherein the abundance of one or more cytosolic proteins is depleted by at least about 90% as compared to the cells from which the organelle complexes population are derived, optionally the cytosolic proteins are p70S6K and/or glyceraldehyde 3-phosphate dehydrogenase (GAPDH).

11. The organelle complexes population of any one of claims 1-10, wherein the organelle complexes comprise:
one or more mitochondrial matrix proteins, optionally mitochondrial transcription factor A (TFAM) and/or citrate synthase (CS);
one or more outer mitochondrial membrane proteins, optionally outer mitochondrial membrane complex subunit 20 (TOMM20);
one or more lysosome proteins, optionally lysosomal-associated membrane protein 2 (LAMP2), mannose-6-phosphate receptor (M6PR), and/or lysosomal-associated membrane protein 1 (LAMP1);
one or more peroxisome proteins, optionally catalase and/or ATP-binding cassette transporter 1, subfamily D, type 3 (ABCD3);
one or more Golgi apparatus proteins, optionally Golgin-97, Sintaxin-6, TGOLN2/trans-Golgi network protein 2 (TGN46), Golgi matrix protein 130 (GM130), and/or Mannosidase Alpha Class 2A Member 1 (MAN2A1); and/or
one or more endoplasmic reticulum proteins, optionally Calreticulin and/or Calnexin.

12. The organelle complexes population of any one of claims 1-11, wherein the organelle complexes population comprises first organelle complexes, or a combination of first organelle complexes and second organelle complexes,
wherein first organelle complexes are derived from (i) frozen cells; (ii) floating cells; and/or (iii) cells contacted with a surfactant at a concentration at or above the critical micellar concentration (CMC) for the surfactant, and
wherein second organelle complexes are derived from (i) adherent cells; and/or (ii) cells contacted with a surfactant at a concentration below the critical micellar concentration (CMC) for the surfactant.

13. The organelle complexes population of any one of claims 1-12, wherein:
first organelle complexes comprise at least about 1.1-fold more of one or more lysosome proteins as compared to second organelle complexes, optionally lysosomal-associated membrane protein 2 (LAMP2), mannose-6-phosphate receptor (M6PR), and/or lysosomal-associated membrane protein 1 (LAMP1);
first organelle complexes comprise at least about 1.1-fold more of one or more peroxisome proteins as compared to second organelle complexes, optionally catalase and/or ATP-binding cassette transporter 1, subfamily D, type 3 (ABCD3);
first organelle complexes comprise at least about 1.1-fold more of one or more Golgi apparatus proteins as compared to second organelle complexes, optionally Golgin-97, Sintaxin-6, TGOLN2/trans-Golgi network protein 2 (TGN46), Golgi matrix protein 130 (GM130), and/or Mannosidase Alpha Class 2A Member 1 (MAN2A1);
first organelle complexes comprise at least about 1.1-fold more of one or more endoplasmic reticulum proteins as compared to second organelle complexes, optionally Calreticulin and/or Calnexin; and/or
second organelle complexes comprise at least about 1.1-fold more of one or more cytosolic proteins as compared to first organelle complexes, optionally p70S6K and/or glyceraldehyde 3-phosphate dehydrogenase (GAPDH).

14. The organelle complexes population of any one of claims 1-13, wherein upon contact of the organelle complexes with a population of cells, the organelle complexes are capable of incorporating into the cells, optionally at least about 2-fold more organelle complexes are capable of incorporating into the cells as compared to a population of homogenized mitochondria.

15. The organelle complexes population of any one of claims 1-14, wherein upon contact of the organelle complexes with a population of host cells, the organelle complexes have superior incorporation capability into host cells, as compared to a population of homogenized mitochondria.

16. The organelle complexes population of any one of claims 1-15, wherein upon contact of first organelle complexes with a population of host cells, the first organelle complexes have superior incorporation capability into host cells, as compared to second organelle complexes.

17. The organelle complexes population of any one of claims 1-16, wherein upon contact of the organelle complexes with a population of host cells, the organelle complexes have superior incorporation capability into host cells, optionally at least 2-fold more organelle complexes are capable of incorporation into host cells as compared to a population of homogenized mitochondria.

18. The organelle complexes population of any one of claims 1-17, wherein the mitochondria of the organelle complexes are capable of incorporation into cells after the population undergoes one or more freeze-thaw cycles, optionally at least 2-fold more mitochondria of the organelle complexes are capable of incorporation into cells after the population undergoes one or more freeze-thaw cycles as compared to a population of homogenized mitochondria.

19. The organelle complexes population of any one of claims 1-18, wherein at least about 80% of the organelle complexes in the population are between about 500 nm and about 3500 nm in size, optionally about 200 nm to about 1000 nm in size.

20. The organelle complexes population of any one of claims 1-19, wherein the organelle complexes population are derived from cells treated with a mitochondria-activating agent, optionally resveratrol.

21. A composition comprising the organelle complexes population of any one of claims 1-20.

22. A formulation comprising the composition of claim 21 and a pharmaceutically acceptable carrier.

23. A method for generating first organelle complexes population, the method comprising:
incubating cells in a first solution comprising a surfactant at a first temperature;
removing the surfactant to form a second solution; and
recovering first organelle complexes from the second solution,
wherein the first organelle complexes comprise mitochondria and one or more of endoplasmic reticulum, peroxisomes, lysosomes, and Golgi apparatus,
wherein the first organelle complexes population are depleted of cytosolic macromolecules, and wherein:
(i) cells in the first solution are incubated with the surfactant at a concentration at or above the critical micellar concentration (CMC) for the surfactant; and/or
(ii) the cells comprise or are derived from floating cells or frozen cells.

24. The method of claim 23, wherein the method comprises incubating the second solution at a second temperature.

25. The method of any one of claims 23-24, wherein the first organelle complexes comprise mitochondria and two, three, or four of endoplasmic reticulum, peroxisomes, lysosomes, and Golgi apparatus.

26. The method of any one of claims 23-25, wherein the cells in the first solution are contacted with the surfactant at a concentration at least about 5% above the critical micellar concentration (CMC) for the surfactant.

27. The method of any one of claims 23-26, wherein the surfactant is saponin, optionally the surfactant is present at a concentration of about 50 ug/mL.

28. The method of any one of claims 23-27, wherein the surfactant is a nonionic surfactant.

29. The method of any one of claims 23-28, wherein the surfactant is selected from the group consisting of Triton-X 100, Triton-X 114, Nonidet P-40, n-Dodecyl-D-maltoside, Tween-20, Tween-80, saponin, and digitonin.

30. The method of any one of claims 23-29, wherein the first solution further comprises a buffer, optionally comprising one or more of a tonicity agent, osmotic modifier, and a chelating agent.

31. The method of any one of claims 23-30, wherein the first solution comprises a Tris buffer, sucrose, and/or a chelator.

32. The method of any one of claims 23-31, wherein incubating cells in the first solution comprises incubating the cells in the first solution for about 1 minute to about 120 minutes, optionally for about 30 minutes.

33. The method of any one of claims 23-32, wherein the first temperature and/or the second temperature is about 0° C. to about 50° C., optionally the first temperature is 25° C. and the second temperature is about 0° C. to about 4° C.

34. The method of any one of claims 23-33, wherein removing the surfactant comprises one or more washes with a buffer, optionally a Tris buffer.

35. The method of any one of claims 24-34, wherein incubating the second solution comprises incubating the second solution for about 1 minute to about 120 minutes, optionally about 20 minutes.

36. The method of any one of claims 23-35, wherein recovering the first organelle complexes from the second solution comprises tangential flow filtration (TFF), optionally TFF is performed: (i) with a low viscosity buffer, optionally said low viscosity buffer reduces the shear rate; (ii) at about 22° C. to about 25° C.; (iii) at a shear rate less than about 2000 sec⁻¹; (iv) below room temperature, optionally at 4° C.; and/or (v) with a buffer comprising human albumin (HA) or recombinant albumin ( ).

37. The method of any one of claims 23-36, wherein recovering the first organelle complexes from the second solution comprises one or more centrifugation steps.

38. The method of any one of claims 23-37, wherein recovering the first organelle complexes from the second solution comprises:
centrifuging the second solution at a first centrifugal force;
collecting the supernatant;
centrifuging the supernatant at a second centrifugal force; and
collecting the pellet to recover the first organelle complexes.

39. The method of any one of claims 23-38, wherein the first centrifugal force and/or the second centrifugal force is about 100 g to about 5000 g, optionally the first centrifugal force is about 500 g and the second centrifugal force is about 3000 g, optionally the centrifuging is performed for about 10 minutes to about 20 minutes.

40. The method of any one of claims 23-39, wherein centrifuging the supernatant at a second centrifugal force comprises centrifuging at 8000 g for about 20 minutes.

41. The method of any one of claims 23-40, wherein incubating cells in the first solution and/or incubating the second solution comprises applying a physical stimulus to the first solution and/or the second solution, respectively, optionally shaking and/or stirring.

42. The method of claim 41, wherein applying a physical stimulus to the first solution and/or the second solution comprises flowing the first solution and/or the second solution through a flow device, wherein said flow device comprises a fluidic channel comprising two or more segments of varying cross-sectional diameters, optionally said cross-sectional diameters range from about 0.8 mm to about 25.4 mm, further optionally said cross-sectional diameters range from about 1.5 mm to 6.5 mm, optionally said flowing through the flow device generates additional flow and/or shear.

43. The method of any one of claims 23-42, wherein the method further comprises freezing the first organelle complexes, optionally in a buffer comprising a cryoprotectant.

44. The method of claim 43, wherein said cryoprotectant comprises human albumin (HA) and/or glycerol.

45. The method of any one of claims 23-44, wherein the method further comprises treating the cells with a mitochondria-activating agent prior to the incubating step, optionally resveratrol.

46. An organelle complexes population obtained by the method according to any one of claims 23-45.

47. A method for treating a disease or disorder, the method comprising:
contacting cells of a subject in need thereof with an effective amount of: (i) the organelle complexes population of any one of claims 1-20 or 46; (ii) the composition of claim 21; and/or (iii) the formulation of claim 22, thereby treating the disease or disorder.

48. A method for treating a disease or disorder associated with mitochondrial dysfunction, the method comprising:
contacting cells of a subject in need thereof with an effective amount of: (i) the organelle complexes population of any one of claims 1-20 or 46; (ii) the composition of claim 21; and/or (iii) the formulation of claim 22, thereby treating a disease or disorder associated with mitochondrial dysfunction.

49. The method of any one of claims 47-48, wherein contacting cells of the subject comprises a route of administration selected from the group comprising intravenous administration, intra-arterial administration, intra-tracheal administration, subcutaneous administration, intramuscular administration, inhalation, intrapulmonary administration, and intra-ocular administration.

50. The method of any one of claims 47-49, wherein the disease or disorder is selected from the group consisting of diabetes (Type I and Type II), metabolic disease, ocular disorders associated with mitochondrial dysfunction, hearing loss, mitochondrial toxicity associated with therapeutic agents, mitochondrial dysfunction associated with Space travel, cardiotoxicity associated with chemotherapy or other therapeutic agents, a mitochondrial dysfunction disorder, and migraine.

51. The method of any one of claims 47-50, wherein the disease or disorder is selected from the group consisting of mitochondrial myopathy, diabetes and deafness (DAD) syndrome, Barth Syndrome, Leber's hereditary optic neuropathy (LHON), Leigh syndrome, NARP (neuropathy, ataxia, retinitis pigmentosa and ptosis syndrome), myoneurogenic gastrointestinal encephalopathy (MNGIE), MELAS (mitochondrial encephalopathy, lactic acidosis, and stroke-like episodes) syndrome, myoclonic epilepsy with ragged red fibers (MERRF) syndrome, Kearns-Sayre syndrome, and mitochondrial DNA depletion syndrome.

52. The method of any one of claims 47-51, wherein the disease or disorder is an ischemia-related disease or disorder, a genetic disorder, an aging disease or disorder, a neurodegenerative condition, a cardiovascular condition, a cancer, an autoimmune disease, an inflammatory disease, a fibrotic disorder, or any combination thereof.

53. The method of any one of claims 47-52, wherein the ischemia-related disease or disorder is selected from the group consisting of cerebral ischemic reperfusion, hypoxia ischemic encephalopathy, acute coronary syndrome, a myocardial infarction, a liver ischemia-reperfusion injury, an ischemic injury-compartmental syndrome, a blood vessel blockage, wound healing, spinal cord injury, sickle cell disease, and reperfusion injury of a transplanted organ.

54. The method of any one of claims 47-53, wherein the neurodegenerative condition is selected from the group consisting of dementia, Friedrich's ataxia, amyotrophic lateral sclerosis, mitochondrial encephalopathy, lactic acidosis, and stroke-like episodes (MELAS), myoclonic epilepsy with ragged red fibers (MERFF), epilepsy, Parkinson's disease, Alzheimer's disease, or Huntington's Disease, Exemplary neuropsychiatric disorders include bipolar disorder, schizophrenia, depression, addiction disorders, anxiety disorders, attention deficit disorders, personality disorders, autism, and Asperger's disease.

55. The method of any one of claims 47-54, wherein the cardiovascular condition is selected from the group consisting of coronary heart disease, myocardial infarction, atherosclerosis, high blood pressure, cardiac arrest, cerebrovascular disease, peripheral arterial disease, rheumatic heart disease, congenital heart disease, congestive heart failure, arrhythmia, stroke, deep vein thrombosis, and pulmonary embolism.

56. The method of any one of claims 47-55, wherein the disease or disorder is acute respiratory distress syndrome (ARDS) or pre-eclampsia or intrauterine growth restriction (IUGR).
